(19) United States
(12) Patent Application Publication
(10) Pub. No.: US 2026/0048124 A1
(43) Pub. Date: Feb. 19, 2026

(54) METHODS FOR TREATING BRAIN CANCER

(71) Applicant: THE REGENTS OF THE UNIVERSITY OF CALIFORNIA, Oakland, CA (US)

(72) Inventors: Robert PRINS, Los Angeles, CA (US); Richard EVERSON, Los Angeles, CA (US); Willy HUGO, Los Angeles, CA (US); Linda LIAU, Los Angeles, CA (US)

(73) Assignee: THE REGENTS OF THE UNIVERSITY OF CALIFORNIA, Oakland, CA (US)

(21) Appl. No.: 19/298,946

(22) Filed: Aug. 13, 2025

Related U.S. Application Data
(60) Provisional application No. 63/682,711, filed on Aug. 13, 2024.

(51) Int. Cl. A61K 40/19; A61K 40/24

(52) U.S. Cl. CPC A61K 40/19; A61K 40/24

(57) ABSTRACT

The current disclosure provides for a method for stratifying patients based on their predicted efficacy to an immunotherapy. Accordingly, provided herein is a method for treating a subject for brain cancer, the method comprising administering to the subject an immunotherapy, wherein the subject has been determined be negative or low for an interferon gamma activation response in a biological sample from the patient. Also described is a method for predicting patient outcomes and/or for predicting the effectiveness of an immunotherapy for treating brain cancer in a subject in need thereof, the method comprising determining an interferon gamma activation response from a biological sample from the subject. Also provided is a method for evaluating a subject having brain cancer, the method comprising determining an interferon gamma activation response in a biological sample from the subject.

This application claims benefit of priority of U.S. Provisional Application No. 63/682,711, filed Aug. 13, 2024, which is hereby incorporated by reference in its entirety.

This invention was made with government support under CA123396, and CA211015 awarded by the National Institutes of Health. The government has certain rights in the invention.

### BACKGROUND

### I. Field of the Invention

The present invention relates generally to the fields of molecular biology and therapeutic diagnosis. More particularly, it concerns methods for treating subjects with and monitoring subjects on immunotherapy.

### II. Background

There have been significant advances in the genetic and immunologic understanding of primary brain tumors, such as malignant gliomas. Yet, it has still proven difficult to improve long-term outcomes in patients using standard of care therapies. It has been demonstrated that autologous tumor lysate (ATL) dendritic cell (DC) vaccination can induce local and systemic anti-tumor immune responses in malignant glioma patients, and clinical trials have suggested that this may extend survival in this deadly condition. However, variable response rates in cancer immunotherapy trials have prompted the search for potential strategies to identify factors and signatures of patients that may predict an efficacious and/or non-toxic response to the immunotherapy. Therefore, there is a need in the art for improved methods for treating subjects with immunotherapies.

### SUMMARY

The current disclosure provides for a method for stratifying patients based on their predicted efficacy to an immunotherapy. Accordingly, provided herein is a method for treating a subject for brain cancer, the method comprising administering to the subject an immunotherapy, wherein the subject has been determined be negative or low for an interferon activation response in a biological sample from the subject. Also described is a method for predicting subject outcomes and/or for predicting the effectiveness of an immunotherapy for treating brain cancer in a subject in need thereof, the method comprising determining an interferon activation response from a biological sample from the subject. Also provided is a method for evaluating a subject having brain cancer, the method comprising determining an interferon activation response in a biological sample from the subject. A further aspect relates to a method for treating a subject having brain cancer and that has been previously treated with a neoadjuvant cancer vaccine, the method comprising administering an adjuvant immunotherapy to the subject; wherein the subject has been determined to have a positive interferon activation response in a biological sample from the subject; wherein the immunotherapy comprises immune checkpoint inhibitor (ICI) therapy; and wherein the cancer vaccine comprises ATL-DC vaccine or neoantigen therapy.

The brain cancer may comprise or exclude glioma. The glioma may comprise or exclude Grade 3 or 4 gliomas. The glioma may comprise or exclude anaplastic glioma, astrocytoma, ependymomas, oligodendroglioma, glioblastoma, or mixed glioma. The cancer may comprise or exclude IDH wildtype or IDH-mutated cancer. The subject may comprise or exclude one that has been determined to have IDH wildtype or IDH-mutated cancer. The cancer may comprise or exclude MGMT methylation positive or MGMT methylation negative cancer. The subject may comprise or exclude one that has been determined to have MGMT methylation positive or MGMT methylation negative cancer. The cancer may comprise or exclude EGFR amplification positive or EGFR amplification negative cancer. The subject may comprise or exclude one that has been determined to have EGFR amplification positive or EGFR amplification negative cancer. The subject may comprise or exclude one that has undergone surgical resection of the tumor.

The subject may comprise or exclude one that has been previously treated with an anti-cancer therapy. The anti-cancer therapy may be a neoadjuvant therapy. The anti-cancer therapy may comprise or exclude a TLR agonist and/or an immunotherapy. The subject may comprise or exclude one that been treated, will be treated, or is being treated with one or both of a TLR agonist and an immunotherapy. The immunotherapy may comprise or exclude a cancer vaccine. The cancer vaccine may comprise or exclude a dendritic cell (DC) vaccine and/or a neoantigen vaccine. The DC vaccine may comprise an autologous tumor lysate (ATL) DC vaccine. The immunotherapy may be an adjuvant immunotherapy. The immunotherapy may be a neoadjuvant immunotherapy. The immunotherapy may comprise or exclude immune checkpoint inhibitor (ICI) therapy. The ICI therapy may comprise or exclude a mono- or a combination-ICI therapy. The ICI therapy may comprise or exclude an inhibitor of PD-1, PDL1, PDL2, CTLA-4, B7-1, and/or B7-2. The ICI therapy may comprise or exclude an anti-PD-1 antibody, and anti-PDL1 antibody, and/or an anti-CTLA-4 antibody. The ICI therapy may comprise or exclude one or more of nivolumab, atezolizumab, avelumab, durvalumab, cemiplimab, pembrolizumab, pidilizumab, ipilimumab or tremelimumab.

The TLR agonist may comprise or exclude a TLR3, TLR7, TLR8, TLR9, and/or TLR10 TLR agonist. The TLR agonist may comprise or exclude a TLR3 agonist. The TLR3 agonist may comprise or exclude polyinosinic acid polycytidylic acid stabilized with polylysine (poly-ICLC). The TLR agonist may comprise or exclude a TLR7 or TLR7/8 agonist. The TLR agonist may comprise or exclude Imiquimod or Resiquimod.

The biological sample may comprise or exclude peripheral blood mononuclear cells (PBMCs). The biological sample may comprise or exclude whole blood or a fraction thereof. The biological sample may comprise or exclude urine, blood, feces, or saliva. The biological sample may comprise or exclude a biological sample described herein. The biological sample may be from a subject that has received a cancer vaccine. The biological sample may be from a subject that received a cancer vaccine at a time point of 0-24 hours prior to the collection of the biological sample. The biological sample may have been collected from the subject at a time point of, of at least, or of at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 hours, days, or weeks, or any derivable range therein.

The interferon activation response may comprise a GSVA (gene set variation analysis) score of less than 0 for a IFNg (interferon gamma) or IFNa (interferon alpha) gene set. The GSVA score, or a positive GSVA score, or a negative GSVA score, or a high GSVA score, or a low GSVA score may be, be at least, may be at most, or may be exactly −50, −49.8, −49.6, −49.4, −49.2, −49, −48.8, −48.6, −48.4, −48.2, −48, −47.8, −47.6, −47.4, −47.2, −47, −46.8, −46.6, −46.4, −46.2, −46, −45.8, −45.6, −45.4, −45.2, −45, −44.8, −44.6, −44.4, −44.2, −44, −43.8, −43.6, −43.4, −43.2, −43, −42.8, −42.6, −42.4, −42.2, −42, −41.8, −41.6, −41.4, −41.2, −41, −40.8, −40.6, −40.4, −40.2, −40, −39.8, −39.6, −39.4, −39.2, −39, −38.8, −38.6, −38.4, −38.2, −38, −37.8, −37.6, −37.4, −37.2, −37, −36.8, −36.6, −36.4, −36.2, −36, −35.8, −35.6, −35.4, −35.2, −35, −34.8, −34.6, −34.4, −34.2, −34, −33.8, −33.6, −33.4, −33.2, −33, −32.8, −32.6, −32.4, −32.2, −32, −31.8, −31.6, −31.4, −31.2, −31, −30.8, −30.6, −30.4, −30.2, −30, −29.8, −29.6, −29.4, −29.2, −29, −28.8, −28.6, −28.4, −28.2, −28, −27.8, −27.6, −27.4, −27.2, −27, −26.8, −26.6, −26.4, −26.2, −26, −25.8, −25.6, −25.4, −25.2, −25, −24.8, −24.6, −24.4, −24.2, −24, −23.8, −23.6, −23.4, −23.2, −23, −22.8, −22.6, −22.4, −22.2, −22, −21.8, −21.6, −21.4, −21.2, −21, −20.8, −20.6, −20.4, −20.2, −20, −19.8, −19.6, −19.4, −19.2, −19, −18.8, −18.6, −18.4, −18.2, −18, −17.8, −17.6, −17.4, −17.2, −17, −16.8, −16.6, −16.4, −16.2, −16, −15.8, −15.6, −15.4, −15.2, −15, −14.8, −14.6, −14.4, −14.2, −14, −13.8, −13.6, −13.4, −13.2, −13, −12.8, −12.6, −12.4, −12.2, −12, −11.8, −11.6, −11.4, −11.2, −11, −10.8, −10.6, −10.4, −10.2, −10, −9.8, −9.6, −9.4, −9.2, −9, −8.8, −8.6, −8.4, −8.2, −8, −7.8, −7.6, −7.4, −7.2, −7, −6.8, −6.6, −6.4, −6.2, −6, −5.8, −5.6, −5.4, −5.2, −5, −4.8, −4.6, −4.4, −4.2, −4, −3.8, −3.6, −3.4, −3.2, −3, −2.8, −2.6, −2.4, −2.2, −2, −1.8, −1.6, −1.4, −1.2, −1, −0.8, −0.6, −0.4, −0.2, 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 30, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, or 50, or any range derivable therein. Gene set variation analysis is known in the art and described in, for example, Hanzelmann et al. BMC Bioinformatics 2013, 14:7, which is herein incorporated by reference for all purposes.

The GSVA score may be a score based on the interferon gene set of Table 3, 4, or 5 or any combination of the genes therein.

TABLE 3
HALLMARK INTERFERON GAMMA RESPONSE

ADAR
APOL6
ARID5B
ARL4A
AUTS2
B2M
BANK1
BATF2
BPGM
BST2
BTG1
C1R
C1S
CASP1
CASP3
CASP4
CASP7
CASP8
CCL2
CCL5
CCL7
CD274
CD38
CD40
CD69
CD74
CD86
CDKN1A
CFB
CFH
CIITA
CMKLR1
CMPK2
CMTR1
CSF2RB
CXCL10
CXCL11
CXCL9
DDX58
DDX60
DHX58
EIF2AK2
EIF4E3
EPSTI1
FAS
FCGR1A
FGL2
FPR1
FTSJD2
GBP4
GBP6
GCH1
GPR18
GZMA
HELZ2
HERC6
HIF1A
HLA-A
HLA-B
HLA-DMA
HLA-DQA1
HLA-DRB1
HLA-G
ICAM1
IDO1
IFI27
IFI30
IFI35
IFI44
IFI44L
IFIH1
IFIT1
IFIT2
IFIT3
IFITM2
IFITM3
IFNAR2
IL10RA
IL15
IL15RA
IL18BP
IL2RB
IL4R
IL6
IL7
IRF1
IRF2
IRF4
IRF5
IRF7
IRF8
IRF9
ISG15
ISG20
ISOC1
ITGB7
JAK2
KLRK1
LAP3
LATS2
LCP2
LGALS3BP
LY6E
LYSMD2
MARCHF1
METTL7B
MT2A
MTHFD2
MVP
MX1
MX2
MYD88
NAMPT
NCOA3
NFKB1
NFKBIA
NLRC5
NMI
NOD1
NUP93
OAS2
OAS3
OASL
OGFR
P2RY14
PARP12
PARP14
PDE4B
PELI1
PFKP
PIM1
PLA2G4A
PLSCR1
PML
PNP
PNPT1
PRIC285
PSMA2
PSMA3
PSMB10
PSMB2
PSMB8
PSMB9
PSME1
PSME2
PTGS2
PTPN1
PTPN2
PTPN6
RAPGEF6
RBCK1
RIGI
RIPK1
RIPK2
RNF213
RNF31
RSAD2
RTP4
SAMD9L
SAMHD1
SECTM1
SELP
SERPING1
SLAMF7
SLC25A28
SOCS1
SOCS3
SOD2
SP110
SPPL2A
SRI
SSPN
ST3GAL5
ST8SIA4
STAT1
STAT2
STAT3
STAT4
TAP1
TAPBP
TDRD7
TNFAIP2
TNFAIP3
TNFAIP6
TNFSF10
TOR1B
TRAFD1
TRIM14
TRIM21
TRIM25
TRIM26
TXNIP
UBE2L6
UPP1
USP18
VAMP5
VAMP8
VCAM1
WARS1
XAF1
XCL1
ZBP1
ZNFX1

The interferon gamma activation response may be based on the Human Gene Set: HALLMARK_INTERFERON_GAMMA_RESPONSE, which is known in the art and can be found, for example, on the world wide web at gsea-msigdb.org/gsea/msigdb/cards/HALLMARK_INTERFERON_GAMMA_RESPONSE. A further interferon activation response may be based on the following gene set: MOSERLE_IFNA_RESPONSE. This is demonstrated in Tables 4 and 5. A negative or low interferon activation response indicates a favorable subject outcome and positive or high interferon activation response indicates an unfavorable subject outcome. The method may further comprise treating the subject determined to have a negative or low interferon activation response with an immunotherapy.

TABLE 4
MOSERLE_IFNA_RESPONSE

| | GENE | |
|---|---|---|
| PROBE | SYMBOL | GENE_TITLE |
| RTP4 | RTP4 | receptor transporter protein 4 |
| CD274 | CD274 | CD274 molecule |
| IFIT3 | IFIT3 | interferon-induced protein with |
| | | tetratricopeptide repeats 3 |
| CXCL10 | CXCL10 | chemokine (C-X-C motif) ligand 10 |
| STAT1 | STAT1 | signal transducer and activator of |
| | | transcription 1, 91 kDa |
| IFIT1 | IFIT1 | interferon-induced protein with |
| | | tetratricopeptide repeats 1 |
| TNFSF10 | TNFSF10 | tumor necrosis factor (ligand) superfamily, |
| | | member 10 |
| IFIT2 | IFIT2 | interferon-induced protein with |
| | | tetratricopeptide repeats 2 |
| DDX60 | null | null |
| RSAD2 | RSAD2 | radical S-adenosyl methionine domain |
| | | containing 2 |
| USP18 | USP18 | ubiquitin specific peptidase 18 |
| CMPK2 | null | null |
| SAMD9L | SAMD9L | sterile alpha motif domain containing 9-like |
| DDX58 | DDX58 | DEAD (Asp-Glu-Ala-Asp) box polypeptide |
| | | 58 |
| IFIH1 | IFIH1 | interferon induced with helicase C domain 1 |
| IFITM1 | IFITM1 | interferon induced transmembrane protein 1 |
| | | (9-27) |
| ZC3HAV1 | ZC3HAV1 | zinc finger CCCH-type, antiviral 1 |

TABLE 5
MOSERLE_IFNA_RESPONSE - extended

| | GENE | |
|---|---|---|
| PROBE | SYMBOL | GENE_TITLE |
| RTP4 | RTP4 | receptor transporter protein 4 |
| CD274 | CD274 | CD274 molecule |
| IFIT3 | IFIT3 | interferon-induced protein with |
| | | tetratricopeptide repeats 3 |
| CXCL10 | CXCL10 | chemokine (C-X-C motif) ligand 10 |
| STAT1 | STAT1 | signal transducer and activator of |
| | | transcription 1, 91 kDa |
| IFIT1 | IFIT1 | interferon-induced protein with |
| | | tetratricopeptide repeats 1 |
| TNFSF10 | TNFSF10 | tumor necrosis factor (ligand) superfamily, |
| | | member 10 |
| IFIT2 | IFIT2 | interferon-induced protein with |
| | | tetratricopeptide repeats 2 |
| DDX60 | null | null |
| RSAD2 | RSAD2 | radical S-adenosyl methionine domain |
| | | containing 2 |
| USP18 | USP18 | ubiquitin specific peptidase 18 |
| CMPK2 | null | null |
| SAMD9L | SAMD9L | sterile alpha motif domain containing 9-like |
| DDX58 | DDX58 | DEAD (Asp-Glu-Ala-Asp) box polypeptide |
| | | 58 |
| IFIH1 | IFIH1 | interferon induced with helicase C domain 1 |
| IFITM1 | IFITM1 | interferon induced transmembrane protein 1 |
| | | (9-27) |
| ZC3HAV1 | ZC3HAV1 | zinc finger CCCH-type, antiviral 1 |
| EPSTI1 | EPSTI1 | epithelial stromal interaction 1 (breast) |
| MX1 | MX1 | myxovirus (influenza virus) resistance 1, |
| | | interferon-inducible protein p78 (mouse) |
| GBP1 | GBP1 | guanylate binding protein 1, interferon- |
| | | inducible, 67 kDa |
| IFI44 | IFI44 | interferon-induced protein 44 |
| IFI44L | IFI44L | interferon-induced protein 44-like |
| OAS2 | OAS2 | 2′-5′-oligoadenylate synthetase 2, 69/71 kDa |

The subject may be a human subject. The subject may be a mammal. The subject may be a laboratory test subject. The subject may be a horse, rat, goat, rabbit, mouse, cat, or dog.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments or aspects discussed elsewhere in a different Example or elsewhere in the application, such as in the Summary of Invention, Detailed Description of the Embodiments, Claims, and description of Figure Legends.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects and embodiments of the disclosure as well and vice versa.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1G. Combination of ATL-DC vaccine and TLR agonists results in a robust interferon pathway activation in the patient PBMCs. A, Timeline of PBMC acquisition and analysis using CyTOF and/or RNAseq. V=vaccine, D=Day. B, Schematic of differential gene expression analysis performed on pre-treatment and post-treatment PBMCs of indicated treatment groups. Differentially expressed genes (DEGs) in TLR agonist-treated groups are compared against their changes in the placebo group to identify DEGs specific to the TLR-agonist groups. C, D, Enriched gene set terms in Gene Ontology Biological Process (C) or ARCHS4 TF Coexp (D) datasets that significantly overlap with the union of DEGs from ATL-DC+poly-ICLC and ATL-DC+resiquimod groups (P values, FDR-adjusted, two-sided fisher exact test). E, Differential gene expression (pre vs. post-treatment fold change, in log 2) of representative antigen presentation and IFN related genes across treatment groups (P values, two-sided two-sided Welch t test). F, Gene set enrichment score differences (pre vs. post-treatment, delta GSVA score) of representative IFN related genesets across treatment groups (P values, two-sided Welch t test). For E, F: Each set of three boxes for each chart corresponds to Placebo, Poly-ICLC, and Resiquimod, from left to right, respectively. G, Heatmap of single-sample, gene set enrichment scores (GSVA) of type I and type II interferon genesets in pre-treatment, ATL-DC+placebo, ATL-DC+poly-ICLC and ATL-DC+resiquimod samples.

FIG. 2A-2G. Single cell analysis reveals activation of systemic T cells and monocytes as a part of interferon pathway activation in all myeloid and lymphoid populations. A, A UMAP projection of the pre- and post-treatment PBMC sample pairs from twenty patients (placebo, n=4 pairs; poly-ICLC, n=9 pairs; resiquimod, n=7 pairs). Clustering was performed with a random sampling of 5,000 cells from each patient. B, Heatmap of normalized expression of all 27 cell markers within cell populations identified in the patient PBMCs. C, D, Normalized expression of indicated markers in monocyte (C), or T cell populations (D) within the PBMC samples of patients from indicated treatment groups. P values, two-sided Wilcoxon rank sum test. For C, D: Each set of three boxes for each chart corresponds to Placebo, Poly-ICLC, and Resiquimod, from left to right, respectively. E, UMAP projection of the PBMC-derived single cells (n=99,590). Immune subset associated with each cluster is inferred based on the cluster's differentially expressed transcripts. Canonical markers of known immune subsets are shown. F, G, Heatmaps showing the union of recurrent DEGs computed between ATL-DC treated samples (combined with placebo, resiquimod or poly-ICLC) and pre-treatment samples in the myeloid populations (F) or lymphocyte populations (G). Shown in the heatmaps are the log fold change values of the DEGs in each cell population grouped by their treatment groups.

FIG. 3A-3F. Combined ATL-DC vaccine and TLR agonist treatment show trends of improved tumor control and patient survival. A, B, C, Progression-free survival (PFS, top) and overall survival (OS, bottom) of all patients (A), patient subset with GBM (B), or grade III glioma (C) in indicated treatment groups. P values, log-rank test. D, E, Multivariate Cox proportional hazards analysis assessing the hazard ratios of tumor progression in TLR agonist treatment groups against placebo in all patients (D) or GBM subset (E) after adjusting for other clinical covariates (Tx_Group=treatment group, RecurNum=number of recurrences prior to ATL-DC treatment). F, MR-computed volumes of post-treatment, recurrent tumors in indicated treatment groups. P values, unpaired, two-sided Wilcoxon rank sum test.

FIG. 4A-4C. IFN pathway activation is a positive predictor of survival after ATL-DC vaccine and TLR agonist therapy. A, Kaplan-Meier progression-free survival curves of all patients (left), GBM (center), and Grade III glioma subsets (right) stratified by their HALLMARK_INTERFERON_GAMMA_RESPONSE GSVA scores in their post-treatment PBMCs. P values, log-rank test. B, C, Multivariate Cox proportional hazards analysis assessing hazard in ratios of tumor progression patients with high HALLMARK_INTERFERON_GAMMA_RESPONSE GSVA score of in all patients (B) or GBM subset (C) after adjusting for other clinical covariates.

FIG. 5. CONSORT diagram of clinical trial enrollment.

FIG. 6A-6F. CyTOF and single cell transcriptomics of patient PBMCs before and after ATL-DC vaccine with or without adjuvant TLR agonist. A, Comparison of CD14+ monocyte fraction in post-treatment PBMCs of patients from indicated treatment groups. P values, two-sided Wilcoxon rank sum test. B, Differential gene expression (pre vs. post-treatment fold change, in log 2) of CD14 transcript across treatment groups (P values, two-sided two-sided Welch t test). C, Differential gene expression (pre vs. post-treatment fold change, in log 2) of PDCD1 transcript across treatment groups (P values, two-sided two-sided Welch t test) after adjusting for the change in CD3D transcript expression in the same sample pair. The values approximate the changes of PDCD1 transcript per T cell. D, Normalized expression of indicated markers in CD4 T cell populations within the PBMC samples of patients from indicated treatment groups. P values, two-sided Wilcoxon rank sum test. E, F, Boxplots showing marker gene expressions in lymphoid cell populations (E) or myeloid and proliferative cell populations (F). For 6A-6D: Each set of three boxes for each chart corresponds to Placebo, Poly-ICLC, and Resiquimod, from left to right, respectively.

FIG. 7A-7D. The association between combined ATL-DC vaccine and TLR agonist and patient survival. A, B, Multivariate Cox proportional hazards analysis assessing the hazard ratios of death in TLR agonist treatment groups against placebo in all patients (A) or GBM subset (B) after adjusting for other clinical covariates (Tx_Group=treatment group, RecurNum=number of recurrences prior to ATL-DC treatment; the CoxPH model did not converge when MGMT_methylation was included). C, D, Representative contrast-enhanced MR imaging of patients treated with ATL-DC+poly-ICLC showing initial increase of T2/FLAIR MRI signal (arrows), which either persists or regresses over time. Both patients have significantly longer PFS and OS than the rest of the patients in the cohort.

FIG. 8A-8B. The association between IFN pathway activation and overall survival after ATL-DC vaccine and TLR agonist therapy. A, Kaplan-Meier overall survival curves of all patients (left), GBM (center), and Grade III glioma subsets (right) stratified by their HALLMARK_INTERFERON_GAMMA_RESPONSE GSVA scores in their post-treatment PBMCs. P values, log-rank test. B, Multivariate Cox proportional hazards analysis assessing hazard ratios of death in patients with high HALLMARK_INTERFERON_GAMMA_RESPONSE GSVA score after adjusting for other clinical covariates.

FIG. 9A-9B. Absolute IFN signature of PBMC Samples at Post-Neo is associated with better survival. A, Kaplan-Meier overall survival curves of patients stratified by their MOSERLE_IFNA_RESPONSE GSVA scores in their post-treatment PBMCs; B, Multivariate Cox proportional hazards analysis assessing hazard ratios of death in patients with high MOSERLE_IFNA_RESPONSE GSVA score after adjusting for other clinical covariates.

FIG. 10A-10C. Induction of IFN signature of PBMC Samples at Post-Neo is associated with better survival. A, Kaplan-Meier overall survival curves of patients stratified by their HALLMARK_INTERFERON_GAMMA_RESPONSE GSVA scores in their post-treatment PBMCs; B, Multivariate Cox proportional hazards analysis assessing hazard ratios of death in patients with high HALLMARK_INTERFERON_GAMMA_RESPONSE GSVA score after adjusting for other clinical covariates. Cluster 1: IFN signature induction at post-Neo timepoint (tumor antigen-associated) and Cluster 2: Late IFN signature induction (not tumor antigen-associated); C, HALLMARK_INTERFERON_GAMMA_RESPONSE GSVA scores of cluster 1 and 2 at various timepoints.

### DETAILED DESCRIPTION OF THE INVENTION

There have been significant advances in the genetic and immunologic understanding of primary brain tumors, such as malignant gliomas. Yet, it has still proven difficult to improve long-term outcomes in patients using standard of care therapies. The inventors have demonstrated that autologous tumor lysate (ATL) dendritic cell (DC) vaccination can induce local and systemic anti-tumor immune responses in malignant glioma patients, and clinical trials have suggested that this may extend survival in this deadly condition. However, variable response rates in cancer immunotherapy trials have prompted the search for potential strategies to enhance the immune effects of dendritic cell vaccines. In particular, agonists of a family of pattern-recognition receptors (PRR) called Toll-like receptors (TLR), which appear capable of activating of antigen-presenting cells (i.e., dendritic cells), enhancing T-cell priming, and decreasing myeloid-derived suppressor cells (MDSC), are strong candidates for use in combination with ATL-DC vaccination to potentially enhance the anti-tumor immune response. In addition, the ability to profile such patients with minimally invasive peripheral blood immune monitoring has not, as yet, yielded reliable predictive biomarkers of responsiveness, and represents a significant gap in the understanding of vaccines. The inventors aimed to understand how the adjuvant administration of TLR agonists modified the immune response in in conjunction with ATL-DC vaccination. As such, they conducted a Phase 2 clinical trial in 23 patients diagnosed with newly diagnosed or recurrent WHO Grade III-IV malignant gliomas. The primary objective was to determine the best combination of vaccine and adjuvant, while the secondary objectives included time to tumor progression and overall survival. The inventors performed deep, high-dimensional immune profiling of these patients to understand how TLR agonists may influence the systemic immune responses induced by ATL-DC vaccination. From this immune monitoring, they could identify very significant changes with interferon responsiveness and T cell function/fitness. In addition, the extent of Interferon response gene change was directly related to survival. As such, they believe that DC vaccination together with Poly ICLC may polarize a systemic interferon response, which could represent an important blood biomarker for immunotherapy in this patient population. These results strongly suggest that the changes in Interferon responsiveness following immunotherapy are directly associated with the clinical benefit of immunotherapy in malignant glioma patients irrespective of which combination of vaccine and TLR agonists the patients received. As such, the inventors believe that this Interferon Activation Score is a robust and sensitive biomarker that may be able to predict clinical outcomes in this patient population following immunotherapy.

### I. THERAPEUTIC METHODS

Methods and compositions may be provided for treating subjects based on biomarker levels. Based on a profile of biomarker expression or activity levels, different treatments may be prescribed or recommended for different patients. In some aspects, the methods are for treating a cancer with an immunotherapy, for monitoring a subject being treated with an immunotherapy, or for evaluating a subject for determining whether they should be treated with an immunotherapy.

In some aspects, the cancer is aggressive cancer. In some aspects, the cancer is Stage I cancer. In some aspects, the cancer is Stage II cancer (e.g., IIA, IIB, IIC). In some aspects, the cancer is Stage III cancer (e.g., IIIA, IIIB, IIIC). In some aspects, the cancer is Stage IV cancer (e.g., IVA, IVB). The cancer may be metastatic. The cancer may be recurrent. The cancer may be one that has been determined to be resistant to a therapy described herein.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

Methods may involve the determination, administration, or selection of an appropriate immunotherapy management regimen and predicting the outcome of the same. As used herein the phrase “immunotherapy management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### B. Monitoring

In certain aspects, the biomarker-based method may be combined with one or more other cancer diagnosis or screening tests at increased frequency if the patient is determined to be at high risk for recurrence or have a poor prognosis based on the biomarker as described above.

In some aspects, the methods of the disclosure further include one or more monitoring tests. The monitoring protocol may include any methods known in the art. In particular, the monitoring include obtaining a sample and testing the sample for diagnosis. For example, the monitoring may include endoscopy, biopsy, laparoscopy, colonoscopy, blood test, urine tests, genetic testing, endoscopic ultrasound, X-ray, barium enema x-ray, chest x-ray, barium swallow, a CT scan, a MRI, a PET scan, ultrasound, nuclear medicine (NM) scan, or PET/CT scan. In some aspects, the monitoring test comprises radiographic imaging. Examples of radiographic imaging this is useful in the methods of the disclosure includes renal ultrasound, computed tomographic (CT) scan, magnetic resonance imaging (MRI), body CT scan, NM Mag 3 lasix scan, PET, and body MRI.

### C. ROC Analysis

In statistics, a receiver operating characteristic (ROC), or ROC curve, is a graphical plot that illustrates the performance of a binary classifier system as its discrimination threshold is varied. ROC analysis may be applied to determine a cut-off value or threshold setting of biomarker expression. For example, patients with biological samples determined to have biomarker expression value above a certain cut-off threshold may be determined to have positive or high interferon activation response and patients with biological samples determined to have biomarker expression value below a certain cut-off threshold may be determined to have negative or low interferon activation response. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1−specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

### II. THERAPEUTIC AGENTS

Methods of the disclosure relate to treating subjects and patients with a cancer therapy and/or an additional therapeutic agent. The cancer therapy or additional therapeutic agent may be one described below and may be given with respect to a patient having been determined to have a certain biomarker profile. For example, the therapy described below is given to a patient determined to have a positive or negative interferon activation response. The methods may exclude administration of a therapy below to a subject determined to have a positive or negative an interferon activation response. Also contemplated are combinations of the therapies described below.

### A. Immune Checkpoint Inhibitor (ICI) Therapy

The methods of the disclosure relate to combination therapies with ICI therapy and/or subjects being treated with ICI therapies. Specific ICI therapies are described below.

### 1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### 2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### B. Immunostimulators

The method may comprise or further comprise administration of an additional agent. The additional agent may be an immunostimulator. The term “immunostimulator” as used herein refers to a compound that can stimulate an immune response in a subject, and may include an adjuvant. In some embodiments, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monophosphoryl lipid (MPL) A of Enterobacteria, such as Escherichia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrhoeae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some embodiments, the additional agent comprises an agonist for pattern recognition receptors (PRR), including, but not limited to Toll-Like Receptors (TLRs), specifically TLRs 2, 3, 4, 5, 7, 8, 9 and/or combinations thereof. In some embodiments, additional agents comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some embodiments, the additional agents also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303 (5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an additional agent may be a TLR-4 agonist, such as bacterial lipopolysaccharide (LPS), VSV-G, and/or HMGB-1. In some embodiments, additional agents may comprise TLR-5 agonists, such as flagellin, or portions or derivatives thereof, including but not limited to those disclosed in U.S. Pat. Nos. 6,130,082, 6,585,980, and 7,192,725.

In some embodiments, additional agents may be proinflammatory stimuli released from necrotic cells (e.g., urate crystals). In some embodiments, additional agents may be activated components of the complement cascade (e.g., CD21, CD35, etc.). In some embodiments, additional agents may be activated components of immune complexes. Additional agents also include complement receptor agonists, such as a molecule that binds to CD21 or CD35. In some embodiments, the complement receptor agonist induces endogenous complement opsonization of the synthetic nanocarrier. In some embodiments, immunostimulators are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

### C. Immunotherapies

In some embodiments, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### D. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

### E. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### F. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### G. Chemotherapies

In some embodiments, the additional therapy comprises a chemotherapy. Suitable classes of chemotherapeutic agents include (a) Alkylating Agents, such as nitrogen mustards (e.g., mechlorethamine, cyclophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozotocin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophyllotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluoruracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### H. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

### I. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### J. Other Agents

It is contemplated that other agents may be used in combination with certain aspects of the present embodiments to improve the therapeutic efficacy of treatment. These additional agents include Glucocorticoid therapy, TNF-alpha inhibitors such as infliximab, IFN-gamma inhibitors, IL-6 inhibitors, Mycophenolate mofetil, Cyclosporine, Cyclophosphamide, and Rituximab, and/or other inhibitors of B cell or plasma cell activity and proliferation.

### III. TLR AGONISTS

The TLR agonist may be one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111: B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam2CSK4; lipoarabinomannan or lipomannan from M. smegmatis; triacylated lipoproteins such as Pam3CSK4; lipoproteins such as MALP-2 and MALP-404 from mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLR5 (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™, Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CLO97, R848, or R848 VacciGrade™), TLR9 (e.g., CpG ODNs); and TLR11 (e.g., Toxoplasma gondii Profilin). In certain embodiments, the TLR agonist is a specific agonist listed above. In further embodiments, the TLR agonist is one that agonizes either one TLR or two TLRs specifically.

In some embodiments, the TLR agonist is a TLR7, TLR8, or a TLR7/8 agonist. The TLR agonist may be multiple (polymerized) molecules of the same TLR agonist or may be a mixture of linked different TLR agonists. The TLR agonist may be linked or polymerized by methods known in the art and/or described herein. In some embodiments, the compound (e.g., TLR agonist) is water soluble. Water solubility affects the shelf-life, stability, and pharmaceutical composition of the compound. Due to the structure of TLR7 and TLR8, most TLR7 and/or TLR8 agonists are poorly soluble in water.

### IV. PROTEIN ASSAYS

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis, electrothermal or electrochemical magneto-immunosensors, lateral flow tests strip, Luminex, and enzyme linked immunoabsorbent assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, or immunofluorescence techniques to detect biomarker expression. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73:147-166 (1981).

In some aspects, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some aspects, the antibody need not be labeled, and the presence thereof can be detected using a labeled antibody, which binds to the antibody.

### V. GENE AND RNA EXPRESSION LEVELS

Methods disclosed herein include measuring expression of genes and/or RNAs (RNAs) such as messenger RNAs (mRNAs) and noncoding RNAs (ncRNAs). Measurement of expression can be done by a number of processes known in the art. The process of measuring expression may begin by extracting RNA from a biological sample. Extracted mRNA and/or ncRNA can be detected by hybridization (for example by means of Northern blot analysis or DNA or RNA arrays (microarrays) after converting RNA into labeled cDNA) and/or amplification by means of a enzymatic chain reaction. Quantitative or semi-quantitative enzymatic amplification methods such as polymerase chain reaction (PCR) or quantitative real-time RT-PCR or semi-quantitative RT-PCR techniques or NULISA or bulk RNA sequencing can be used. Suitable primers for amplification methods encompassed herein can be readily designed by a person skilled in the art. Other amplification methods include ligase chain reaction (LCR), transcription-mediated amplification (TMA), strand displacement amplification (SDA), isothermal amplification of nucleic acids, and nucleic acid sequence-based amplification (NASBA). Expression levels of mRNAs and/or ncRNAs may also be measured by RNA sequencing methods known in the art. RNA sequencing methods may include mRNA-seq, total RNA-seq, targeted RNA-seq, small RNA-seq, single-cell RNA-seq, ultra-low-input RNA-seq, RNA exome capture sequencing, and ribosome profiling. Sequencing data may be processed an aligned using methods known in the art.

To normalize the expression values of one gene among different samples, comparing the mRNA and/or ncRNA level of interest in the samples from the subject object of study with a control RNA level is possible. As it is used herein, a “control RNA” is an RNA of a gene for which the expression level does not differ among different non-diseased individuals. In some aspects, the gene may be constitutively expressed in all types of cells. A control RNA is preferably an mRNA derived from a housekeeping gene encoding a protein that is constitutively expressed and carrying out essential cell functions. A known amount of a control RNA may be added to the sample(s) and the value measured for the level of the RNA of interest may be normalized to the value measured for the known amount of the control RNA. Normalization for some methods, such as for sequencing, may comprise calculating the reads per kilobase of transcript per million mapped reads (RPKM) for a gene of interest, or may comprise calculating the fragments per kilobase of transcript per million mapped reads (FPKM) for a gene of interest. Normalization methods may comprise calculating the log 2-transformed count per million (log-CPM). It can be appreciated to one skilled in the art that any method of normalization that accurately calculates the expression value of an RNA for comparison between samples may be used.

Methods disclosed herein may include comparing a measured expression level to a reference expression level. The term “reference expression level” refers to a value used as a reference for the values/data obtained from samples obtained from patients. The reference level can be an absolute value, a relative value, a value which has an upper and/or lower limit, a series of values, an average value, a median, a mean value, or a value expressed by reference to a control or reference value. A reference level can be based on the value obtained from an individual sample, such as, for example, a value obtained from a sample from the subject object of study but obtained at a previous point in time. The reference level can be based on a high number of samples, such as the levels obtained in a cohort of subjects having a particular characteristic. The reference level may be defined as the mean level of the patients in the cohort. The reference may be from subjects that are healthy, subjects without one or more neurological disorder(s), subjects that are age-matched, subjects that are gender-matched, and/or subjects that are race-matched. A reference level can be based on the expression levels of the markers to be compared obtained from samples from subjects who do not have a disease state or a particular phenotype. The person skilled in the art will see that the particular reference expression level can vary depending on the specific method to be performed.

Some embodiments include determining that a measured expression level is higher than, lower than, increased relative to, decreased relative to, equal to, or within a predetermined amount of a reference expression level. In some embodiments, a higher, lower, increased, or decreased expression level is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and some embodiments include determining that the measured expression level is higher by a predetermined amount or lower by a predetermined amount than a reference level. In some embodiments, a level of expression may be qualified as “low” or “high,” which indicates the patient expresses a certain gene or RNA at a level relative to a reference level or a level with a range of reference levels that are determined from multiple samples meeting particular criteria. The level or range of levels in multiple control samples is an example of this. In some embodiments, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criterion or set of criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of gene and/or RNA expression levels to a mean expression level or a reference expression level, the comparison is to be made on a gene-by-gene and RNA-by-RNA basis.

### VI. SAMPLE PREPARATION

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy, or skin biopsy. Alternatively, the sample may be obtained from any other source including but not limited to blood, serum, plasma, urine, pericardial fluid, joint aspiration, pleural fluid, sweat, hair follicle, buccal tissue, tears, menses, feces, or saliva. In certain aspects of the current methods, any medical professional such as a doctor, nurse or medical technician, clinical coordinator may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the check, saliva collection, urine collection, feces collection, collection of menses, tears, any body fluid, blood, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example kidney(s) or related tissues) and one or more samples from another specimen (for example serum, plasma, urine) may be obtained for diagnosis by the methods. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or neurologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### VII. ADMINISTRATION OF THERAPEUTIC COMPOSITIONS

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### VIII. KITS

Certain aspects of the present invention also concern kits containing compositions of the invention or compositions to implement methods of the invention. In some aspects, kits can be used to evaluate one or more biomarkers. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, antibodies, or inhibitors, or any value or range and combination derivable therein. In some aspects, there are kits for evaluating biomarker activity or level in a cell.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1x, 2x, 5x, 10x, or 20x or more.

Kits for using probes, antibodies, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes antibodies that bind to such biomarkers as well as nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, antibodies, probes, and inhibitors are included in some kit aspects. In addition, a kit may include a sample that is a negative or positive control for methylation of one or more biomarkers.

It is contemplated that any method or composition described herein can be implemented with respect to any other method or composition described herein and that different aspects may be combined. The claims originally filed are contemplated to cover claims that are multiply dependent on any filed claim or combination of filed claims.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Dendritic Cell Vaccination in Conjunction with a TLR Agonist Polarizes Interferon Immune Responses in Malignant Glioma Patients

Autologous tumor lysate-pulsed dendritic cell (ATL-DC) vaccination is a promising immunotherapy for patients with high grade gliomas, but responses have not been demonstrated in all patients. To determine the most effective combination of autologous tumor lysate-pulsed DC vaccination, with or without the adjuvant toll-like receptor (TLR) agonists poly-ICLC or resiquimod, the inventors conducted a Phase 2 clinical trial in 23 patients with newly diagnosed or recurrent WHO Grade III-IV malignant gliomas. The inventors then performed deep, high-dimensional immune profiling of these patients to better understand how TLR agonists may influence the systemic immune responses induced by ATL-DC vaccination. Bulk RNAseq data demonstrated highly significant upregulation of type 1 and type 2 interferon gene expression selectively in patients who received adjuvant a TLR agonist together with ATL-DC. CyTOF analysis of patient peripheral blood mononuclear cells (PBMCs) showed increased expression of PD-1 on CD4+ T-cells, decreases in CD38 and CD39 on CD8+ T cells and elevated proportion of monocytes after ATL-DC+TLR agonist administration. In addition, scRNA-seq demonstrated a higher expression fold change of IFN-induced genes with poly-ICLC treatment in both peripheral blood monocytes and T lymphocytes. Patients who had higher expression of interferon response genes lived significantly longer and had longer time to progression compared to those with lower expression. The results suggest that ATL-DC in conjunction with adjuvant poly-ICLC induces a polarized interferon response in circulating monocytes and specific activation of a CD8+ T cell population, which may represent an important blood biomarker for immunotherapy in this patient population.

### A. Introduction

There have been significant advances in the genetic and immunologic understanding of primary brain tumors, such as malignant gliomas. Yet, it has still proven difficult to improve long-term outcomes in patients using standard of care therapies (1). The inventors and others have demonstrated that autologous tumor lysate (ATL) dendritic cell (DC) vaccination can induce local and systemic anti-tumor immune responses in malignant glioma patients, and clinical trials have suggested that this may extend survival in this deadly condition (2-6). However, variable response rates in cancer immunotherapy trials have prompted the search for potential strategies to enhance the immune effects of dendritic cell vaccines. In particular, agonists of a family of pattern-recognition receptors (PRR) called Toll-like receptors (TLR) (7-10), which appear capable of activating of antigen-presenting cells (i.e., dendritic cells), enhancing T-cell priming, and decreasing myeloid-derived suppressor cells (MDSC), are strong candidates for use in combination with ATL-DC vaccination to potentially enhance the anti-tumor immune response. (10,11)

In this study, the inventors report the long-term results of 23 malignant glioma patients enrolled in a phase II randomized clinical trial designed to compare the safety, immune responses, and potential efficacy of ATL-DC vaccination combined with placebo, poly-ICLC, or resiquimod. Post-hoc analysis using cytometry by time-of-flight (CyTOF) and bulk and single-cell RNA sequencing (scRNAseq) technologies were used to detect the cellular and molecular immune signatures from peripheral blood mononuclear cells (PBMCs) pre- and post-treatment.

### B. Results

### 1. Patient Characteristics and Safety

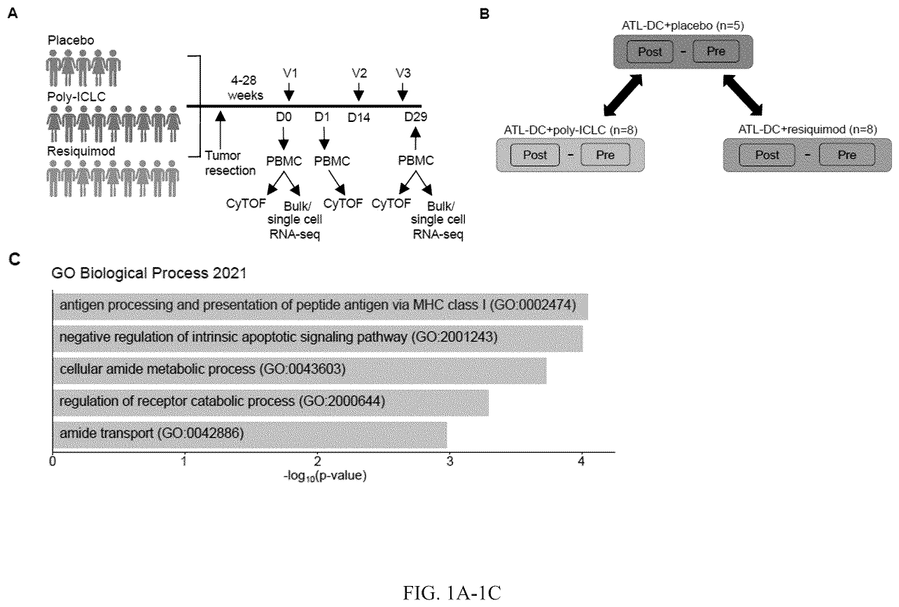

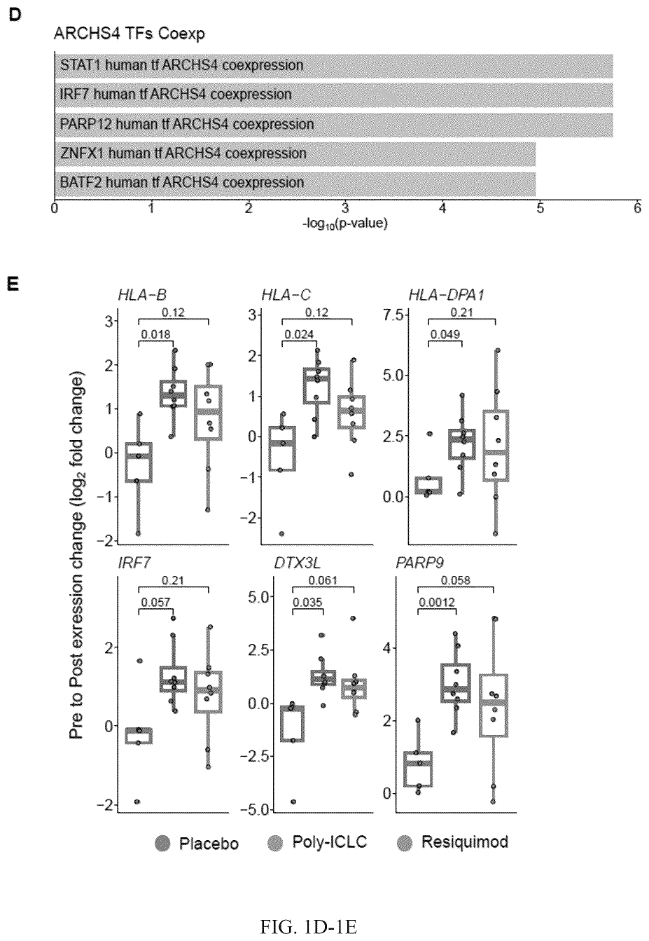

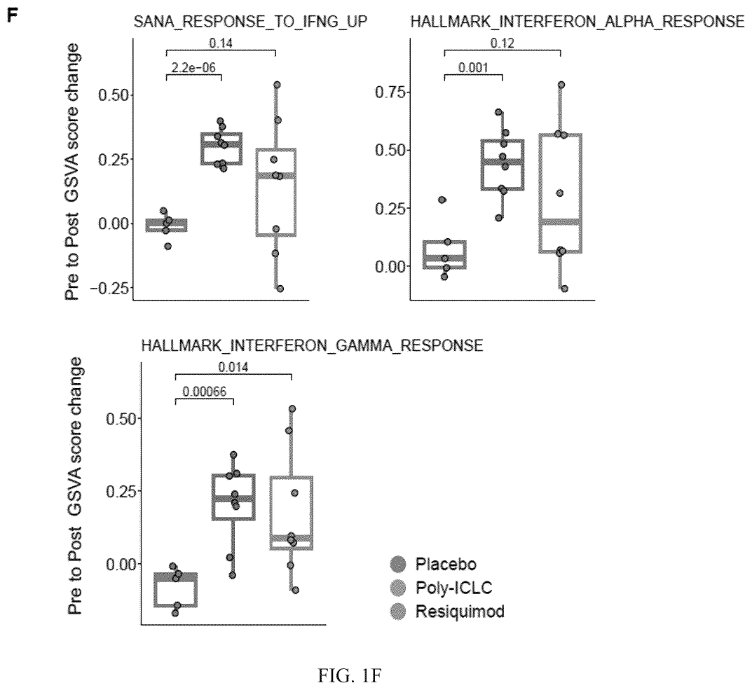

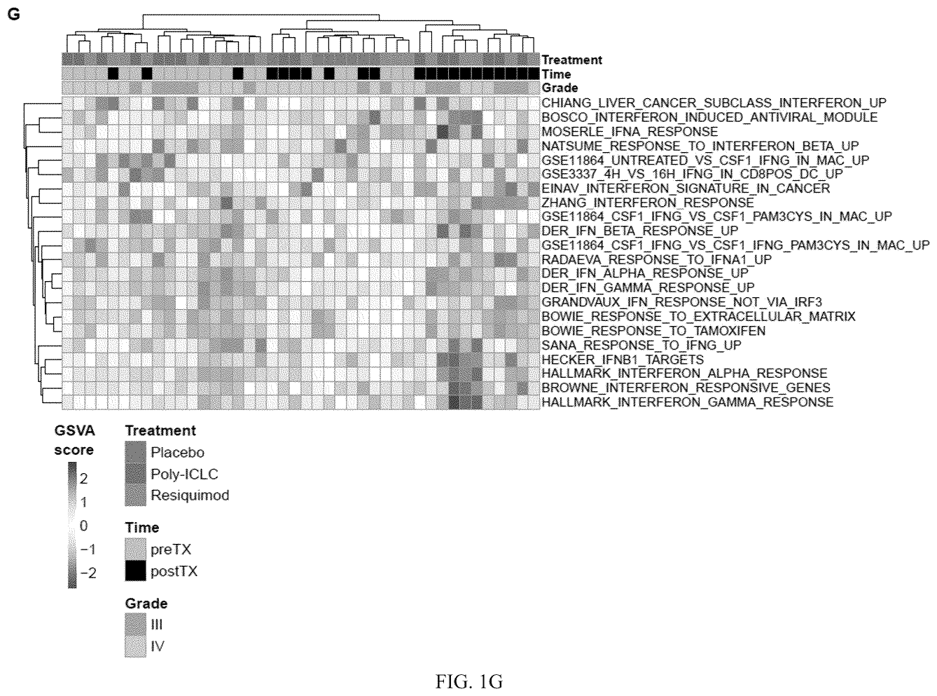

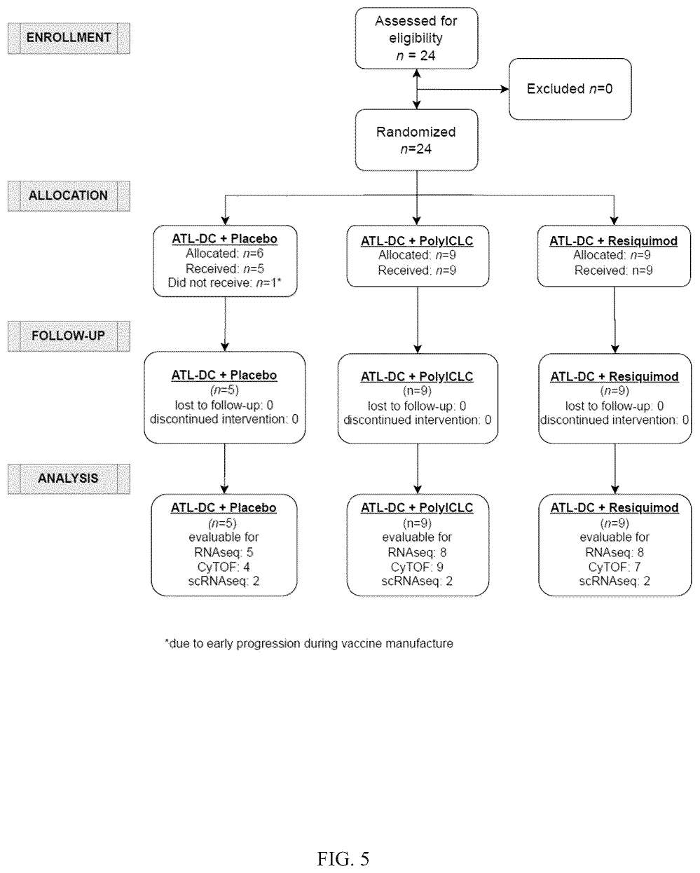

A total of 23 patients with WHO grade III or IV glioma were enrolled and randomized between September 2010 and August 2014. All patients received ATL-DC vaccination. Nine patients were randomized into the adjuvant TLR-7/8 agonist (resiquimod, 3M) group, nine into the adjuvant TLR-3 agonist (poly-ICLC, Oncovir) group, and five received adjuvant placebo (FIG. 1A, FIG. 5). All patients were followed for survival, imaging changes, as well as high dimensional, in-depth systemic immune monitoring. Baseline patient characteristics are presented and segregated by treatment group in Table 1. The median age was 46.6 (S.D. 11.9) years and 57% of the enrolled patients were male. 65% (n=15) had diagnoses of IDH wild type glioblastoma (WHO Grade IV), while 35% (n=8) of the patients had a diagnosis of IDH mutant malignant glioma (WHO Grade III). 52% (n=12) of patients were treated following recurrence, while 48% (n=11) were treated in the newly diagnosed setting. All patients were treated following surgical resection and standard of care treatment. The molecular characteristics of the patient tumors are outlined in Table 1. Overall, MGMT methylation was seen in 35% (n=8), IDH mutations were observed in 35% (n=8, all grade III), and EGFR amplification was seen in 44% (n=10, all glioblastoma) of patients, consistent with the heterogenous population of malignant glioma patients. There were no statistically significant differences in age, sex, Karnofsky performance status, MGMT methylation status, pre- or post-surgery enhancing tumor volume, nor steroid administration at enrollment. No statistically significant differences were observed between the molecular characteristics, although the number of patients in each treatment group was small.

Overall, the addition of a TLR agonist induced only Grade 1-2 treatment-related adverse events (TRAEs), and all adverse events reported resolved without further treatment or hospitalization (Table 2). The most common TRAEs were rash (39%), fever (35%), and fatigue (26%; see Table 2), and were more common in patients treated with resiquimod and poly-ICLC. Other observed adverse events were not uncommon in the setting of post-operative central nervous system (CNS) tumor treatment. Additionally, 88.9% of patients who received resiquimod reported a temporary localized, cutaneous rash that resolved without further treatment. However, no serious adverse events (Grade 3-4) attributable to the treatment were observed. As such, the addition of a TLR agonist to ATL-DC vaccination in malignant glioma patients was found to be safe and tolerable.

### 2. Adjuvant TLR Agonist Treatment Induces Systemic Expression of Type I and Type II Interferon Downstream Genes.

The primary scientific endpoint of this clinical trial was to evaluate the systemic immune response changes induced by ATL-DC vaccination with and without TLR agonist administration. As such, the inventors collected PBMCs at baseline (pre-treatment), one day after the vaccination (on treatment), and then following the completion of the treatment cycle (post-treatment) of each patient (FIG. 1A). The inventors aimed to understand how the adjuvant administration of TLR agonists modified the immune response in comparison with ATL-DC vaccination alone (placebo control). The inventors first performed paired bulk RNA-seq on patient-matched, pre-treatment and post-treatment PBMC samples. For each gene, the inventors computed the difference between its expression in the pre- and post-samples of patients in each treatment group: ATL-DC+placebo (n=5 pairs); ATL-DC+poly-ICLC (n=8 pairs); ATL-DC+resiquimod (n=8 pairs); for brevity, the inventors refer to them as placebo, poly-ICLC and resiquimod, respectively. To identify expression changes specific to the TLR agonist groups, the inventors identified genes whose average upregulation in the TLR agonist pairs (poly-ICLC or resiquimod) were at least two-fold higher than the placebo pairs (FIG. 1B, see Methods).

Genes upregulated in the TLR agonist groups were involved in antigen processing and were enriched with known interferon stimulated genes (ISGs) (FIG. 1C-E). This observation was also confirmed by per-sample gene set enrichment analysis, where the TLR agonist-treated groups displayed higher enrichment of type I and II interferon downstream gene sets compared to ATL-DC/placebo (FIG. 1F). PBMC samples with higher absolute enrichment scores of interferon gene sets were dominated by post-treatment samples from both grade III and IV glioma patients in the TLR-agonist treated groups (FIG. 1G). The inventors noted that the resiquimod group had a more heterogenous response, which resulted in a lower degree of statistical significance compared to that of poly-ICLC group. Nonetheless, the two TLR agonist-treated groups showed a largely similar trend in treatment-induced gene expression changes, which included a measurable increase in the expression of ISGs in the peripheral blood of malignant glioma patients.

### 3. TLR Agonist Treatment Induces Systemic T Cell Activation, Monocyte Proliferation and Interferon Responses in Myeloid and Lymphoid Populations.

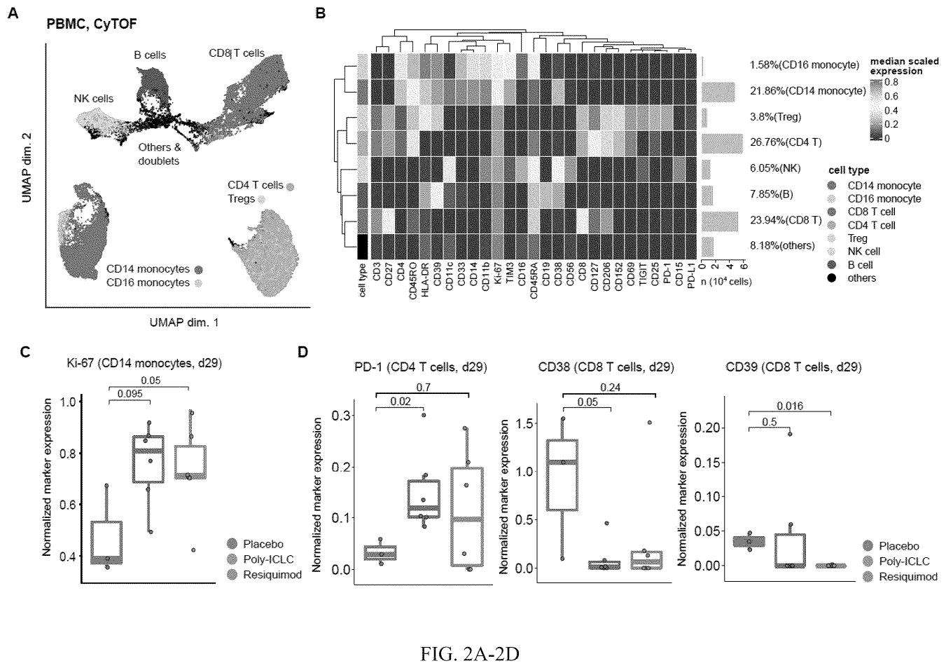

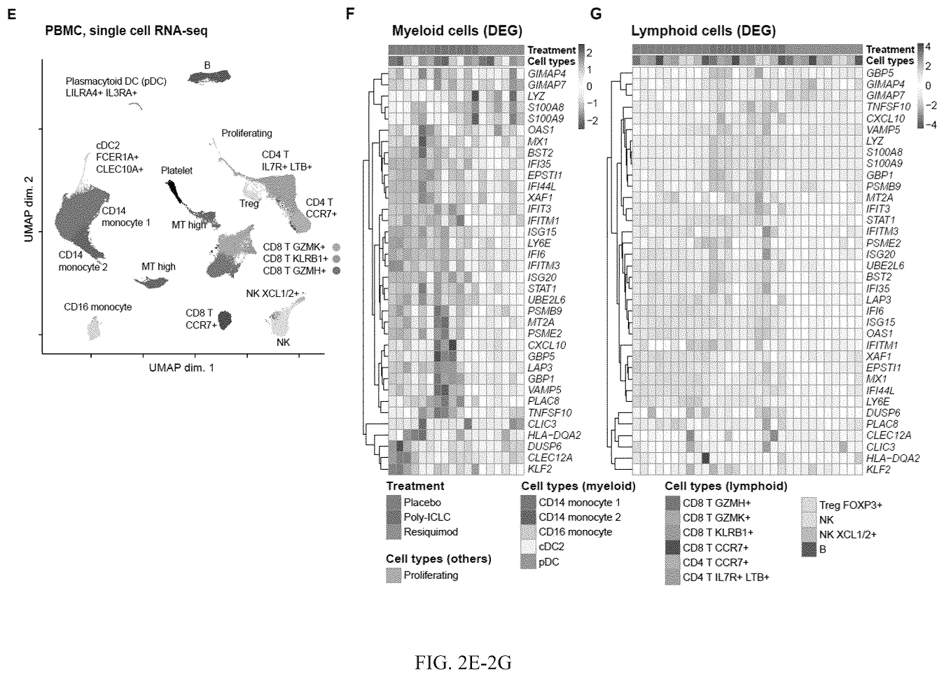

The inventors performed CyTOF on PBMC timepoints with a 27-marker heavy metal antibody-conjugated panel for 20 of the 23 patients (placebo, n=4 pairs; poly-ICLC, n=9 pairs; resiquimod, n=7 pairs). The panel was selected to be able to broadly characterize different immune cell types, activation/effector, memory and exhaustion phenotypes, with a bias towards T-cell relevant markers. The different immune cell type populations were visualized by the uniform manifold approximation and projection (UMAP) method (FIG. 2A), which the inventors broadly assigned to seven different major immune populations based off the normalized heatmap marker expression (FIG. 2B).

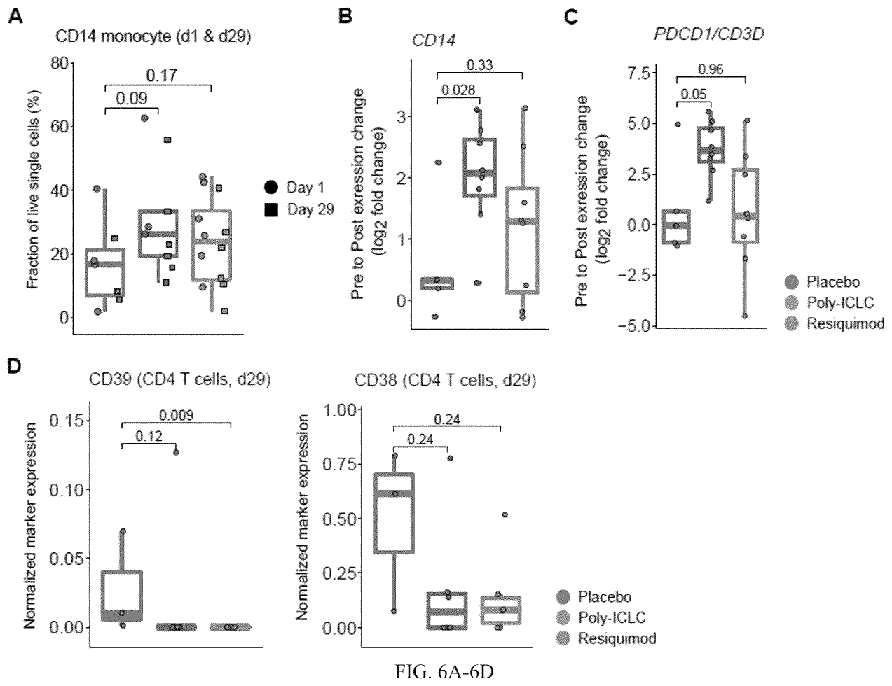

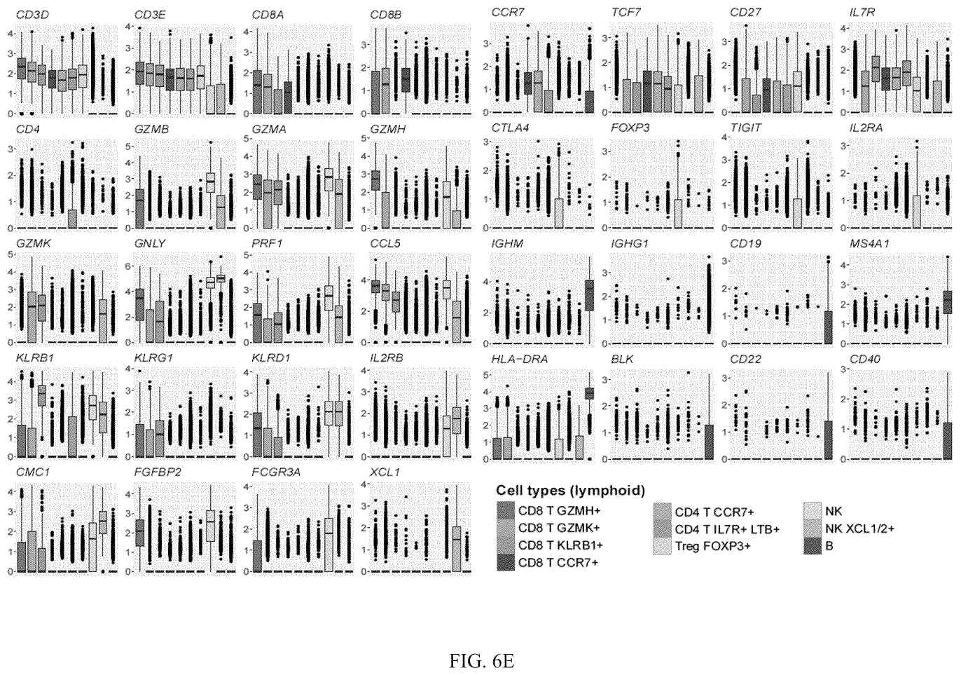

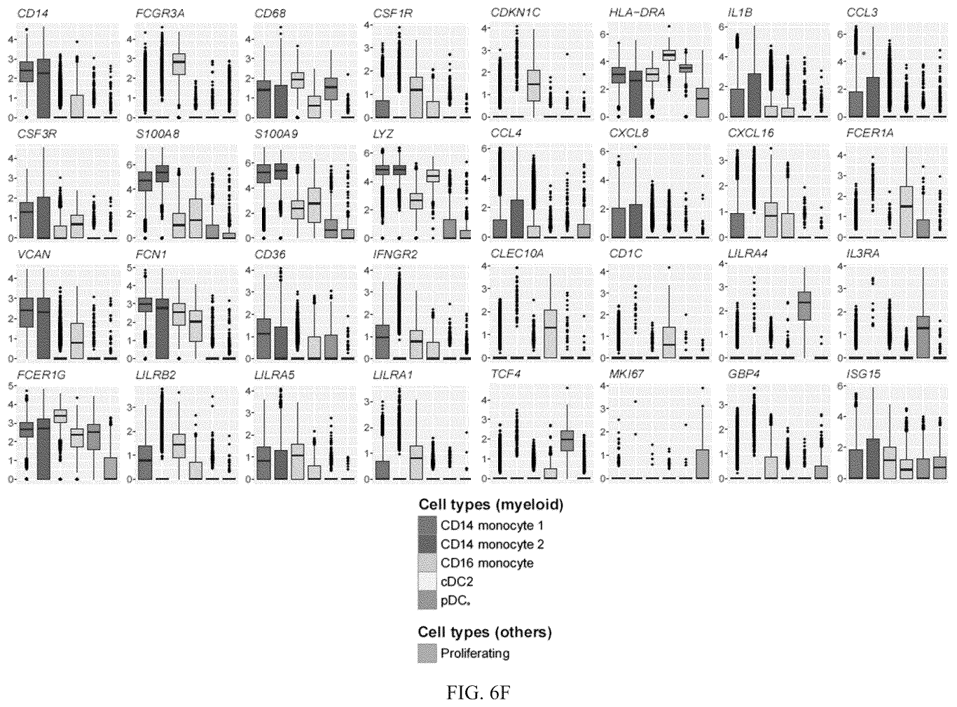

After 3 cycles of treatment, the post-treatment samples of patients in the TLR agonist groups showed a significant increase in the proportion of proliferating Ki67+ CD14+ classical monocytes (FIG. 2C). Such findings were supported by the increased monocyte fraction and CD14 transcript expression after ATL-DC+TLR agonist-treated samples (FIG. 6A, B). ATL-DC+TLR agonist treatment induced PD-1 expression in CD4 T cell population and increased the T-cell normalized expression of PDCD1 (the transcript that encodes PD-1 protein) (FIG. 2D, FIG. 6C). Moreover, expression of markers associated with irreversible T cell exhaustion, such as CD38 and CD39 (33,34), were also reduced after ATL-DC+TLR agonist treatment (FIG. 2D, FIG. 6D). Increased expression of PD-1 and decreased expression of CD38 and CD39 suggest the ATL-DC+TLR agonist combination therapy can improve systemic T cell activity and cellular fitness in the patient.

To delineate the changes induced by ATL-DC and TLR agonist treatment in discrete peripheral blood immune cell subsets, the inventors performed single cell RNA-seq on selected patients at baseline and then following the completion of therapy. The inventors analyzed two representative sample pairs from each cohort (placebo, poly-ICLC, and resiquimod) (data not shown). The inventors identified a total of twelve clusters from the total PBMC immune cell population and annotated these clusters based on differentially expressed gene markers in each cluster. From the initial clustering, the inventors were able to identify multiple populations of CD4+ and CD8+ T cells, two populations of NK cells, three monocytic cell populations, B cell, and dendritic cells (type 2 conventional dendritic cells (cDC2) and plasmacytoid dendritic cells (pDCs), in accordance with the previous characterization of these cell types in peripheral blood (FIG. 2D and FIG. 6E, F).

Differential gene expression analysis across the different lymphoid and myeloid populations revealed concordant upregulation of known ISGs (e.g. IFI6/35/44L, ISG15/20, IFIT3, IFITM1/3, GBP1/5, MX1, STAT1 and CXCL10) and antigen presentation related proteasomes (PSMB9 and PSME2) in both TLR agonist sample pairs. The induction was weaker in the paired PBMC samples obtained from the resiquimod group compared to the poly-ICLC group (FIG. 2F, G)

Thus, the combination of high dimensional proteomics, bulk and single cell RNAseq demonstrates how adjuvant TLR administration in conjunction with ATL-DC reproducibly increases the proportion of canonical CD14+ monocytes within the systemic blood circulation. This TLR agonist administration was also associated with enhanced T cell activity, coupled with decreased expression of CD38 and CD39 and their downstream T cell-suppressive adenosine pathway (33-35). ATL-DC+TLR agonist-driven induction of ISGs across lymphoid and myeloid populations identified in the scRNAseq analysis corroborated the bulk transcriptomic analysis. Given the consistent systemic changes observed with TLR agonist administration, the inventors wondered if there were survival differences between these patient populations.

4. Long-Term Clinical Outcomes of Malignant Glioma Patients Treated with ATL-DC Vaccination Plus Adjuvant TLR Agonists.

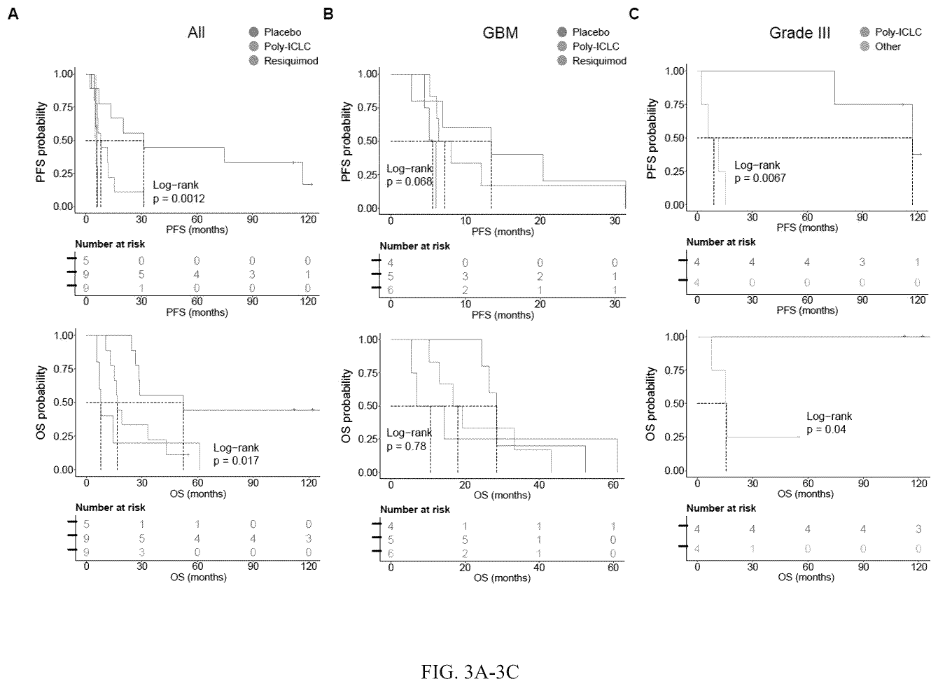

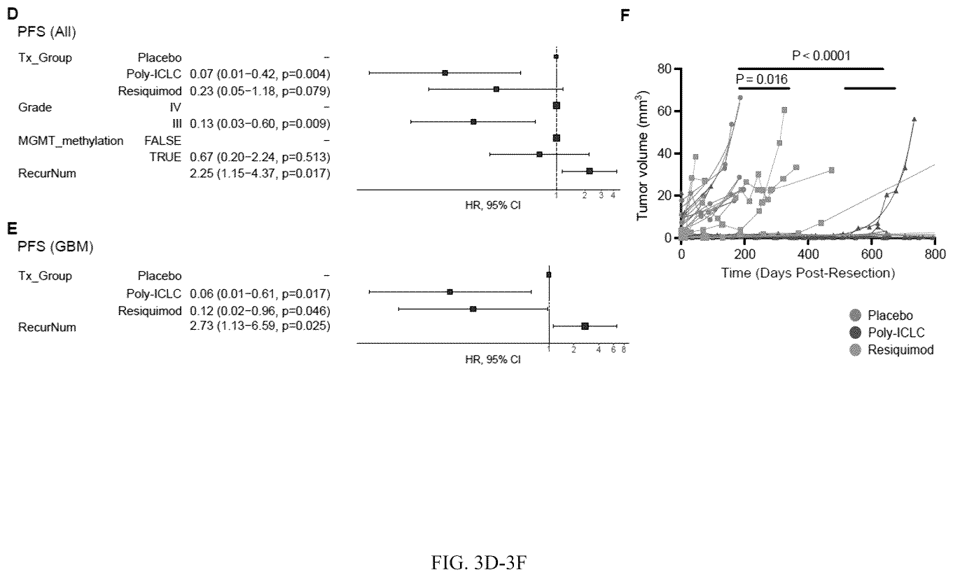

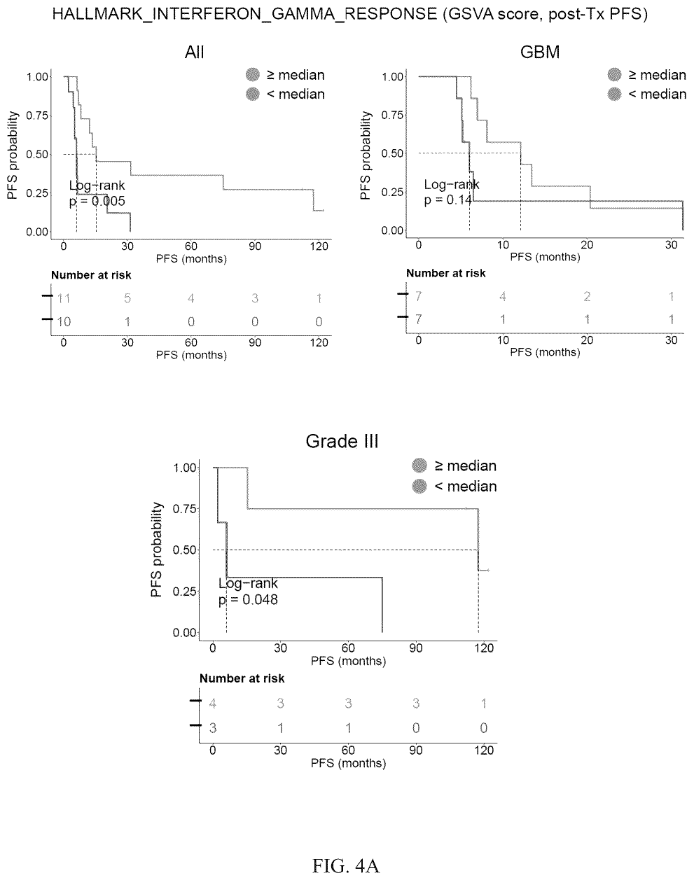

Median follow-up of patients treated on this clinical trial was 2.2 years after surgery, although the long-term survivors have now been followed for over 10 years. Median progression-free survival (PFS) was 8.1 months; and median overall survival (OS) was 26.6 months. Although this clinical trial was not designed to be statistically powered to detect changes in survival between the treatment groups, the inventors nonetheless noted statistically significant differences in median survival between the treatments groups for both OS (placebo: 7.7 months, poly-ICLC: 52.5 months, and resiquimod: 16.7 months; log-rank P=0.017) and PFS (placebo: 5.5 months, poly-ICLC: 31.4 months and resiquimod: 8.1 months; log-rank P=0.0012) (FIG. 3A). Because the trial included patients with both grade III and IV tumors, the inventors stratified the analysis based on tumor grade. When the inventors analyzed only the grade IV (GBM) patients, the inventors observed a trend towards improved PFS (log-rank P=0.068) and OS (P not significant) (FIG. 3B). Interestingly, for the IDH mutant/Grade III cohort, all four patients that received ATL-DC+poly-ICLC treatment are still alive at the data cutoff date (three of the patients have survival >120 months and one >112 months), and they have significantly longer OS and PFS compared to the other grade III patients who received ATL-DC+resiquimod or ATL-DC alone (FIG. 3C).

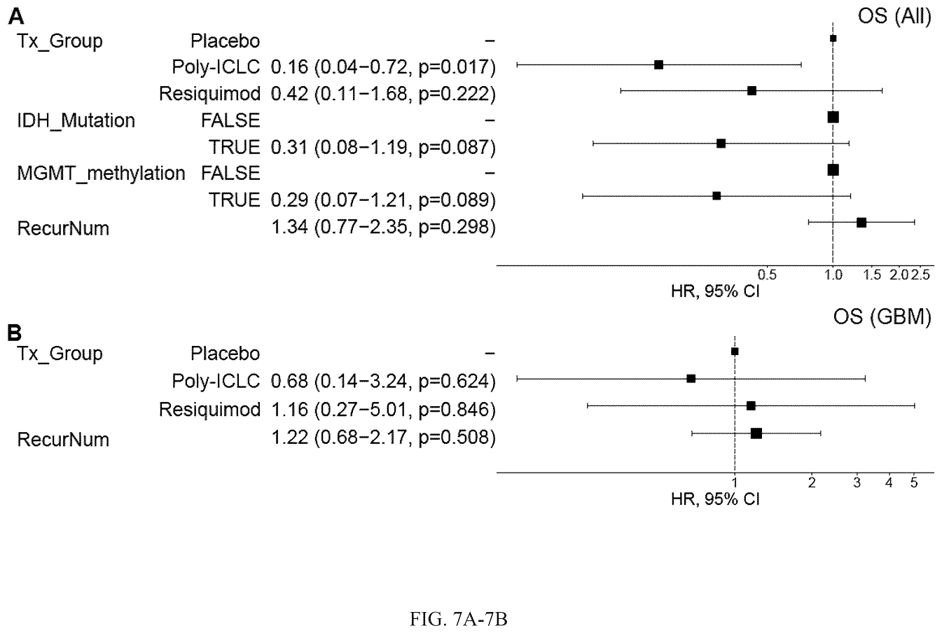

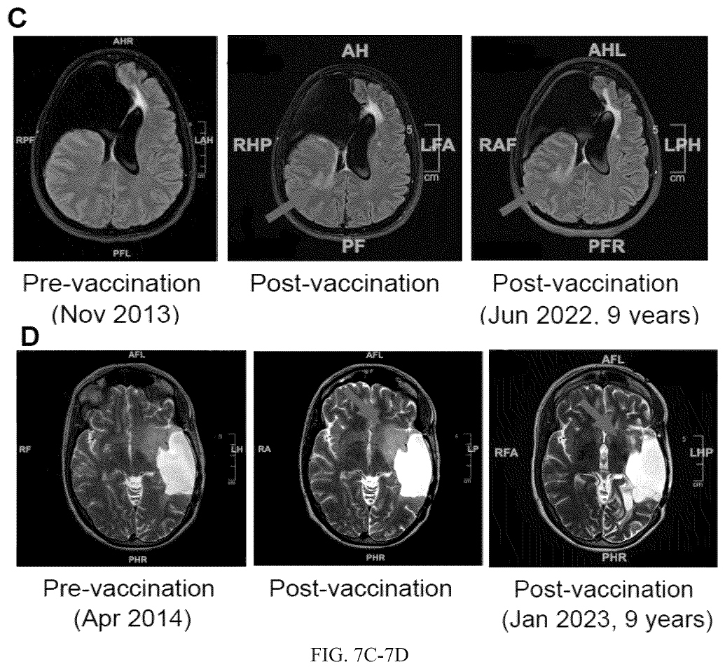

The inventors performed multivariate Cox proportional hazard (PH) analysis, adjusting for clinical variables that are significantly correlated with OS or PFS as a single variable (tumor grade, MGMT methylation status, and number of recurrences). The inventors' analysis confirmed that patients in either the poly-ICLC or resiquimod treatment group had a lower risk of progression that was independent of grade, MGMT methylation, and number of recurrences (FIG. 3D). Risk of death was significantly lower in the poly-ICLC group, while the resiquimod group showed a similar trend that was not statistically significant (FIG. 7A). In the GBM patient subset, TLR agonist treatment also significantly lowered risk of recurrence, but not risk of death (FIG. 3E, FIG. 7B).

To determine whether this treatment directly impacted tumor volume, MR imaging was performed, and contrast-enhancing tumor volume was quantified over time. The inventors noted that the rate of tumor volume increase over time in the ATL-DC/placebo treatment cohort was higher than in the ATL-DC/resiquimod treatment (p=0.022) and the ATL-DC/poly-ICLC treatment groups (P<0.001; FIG. 3F). Interestingly, the inventors observed an increased T2/FLAIR MRI signal after completion of the vaccine series two of the four long-term survivors who received ATL-DC/poly-ICLC (FIG. 7C-D), but such findings are potentially confounded by prior radiation therapy, and thus the inventors cannot ascribe such changes solely to the vaccine/TLR agonist intervention. However, this increased post-vaccination T2/FLAIR on MRI was not seen in patients who did not receive poly-ICLC (not shown).

5. Interferon Activation Score in the Peripheral Blood Immune Cells is a Significant Predictor of Survival after ATL-DC Therapy.

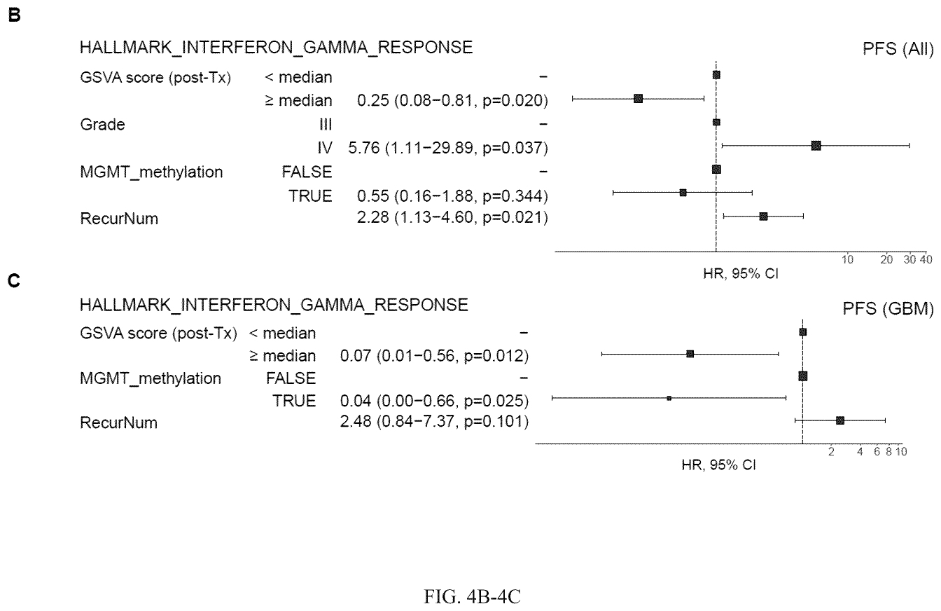

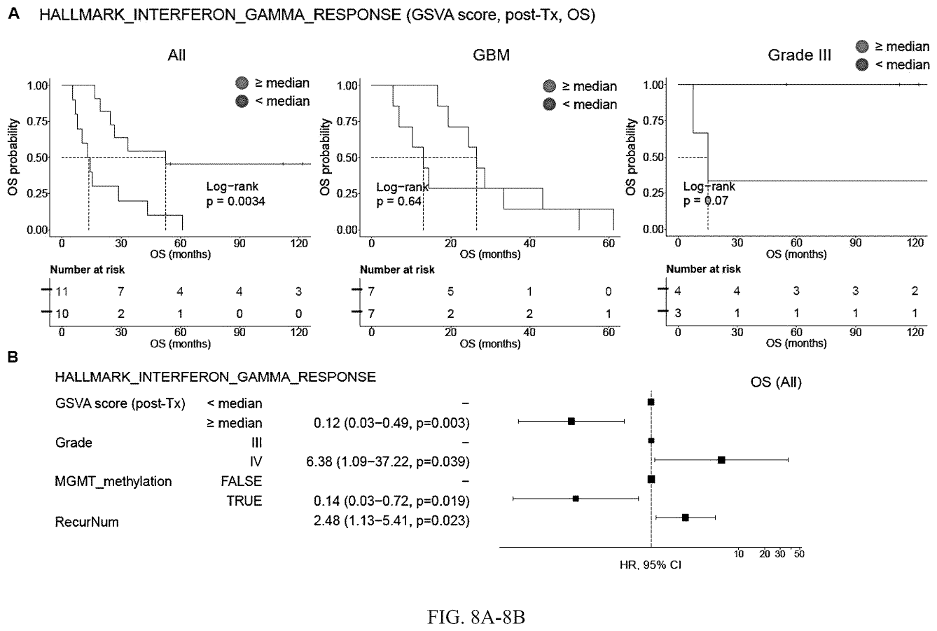

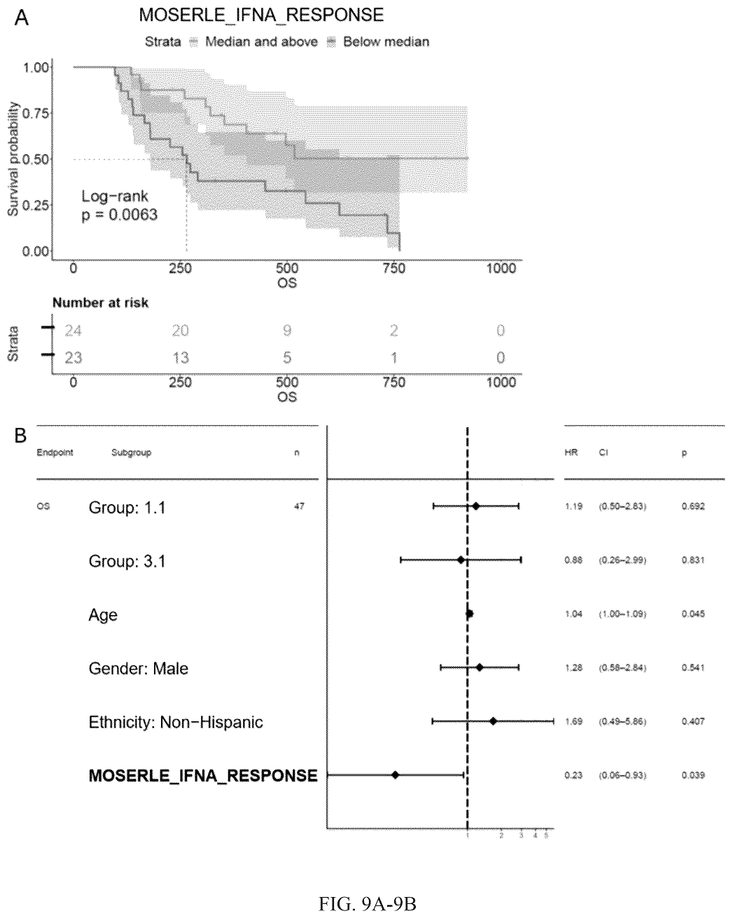

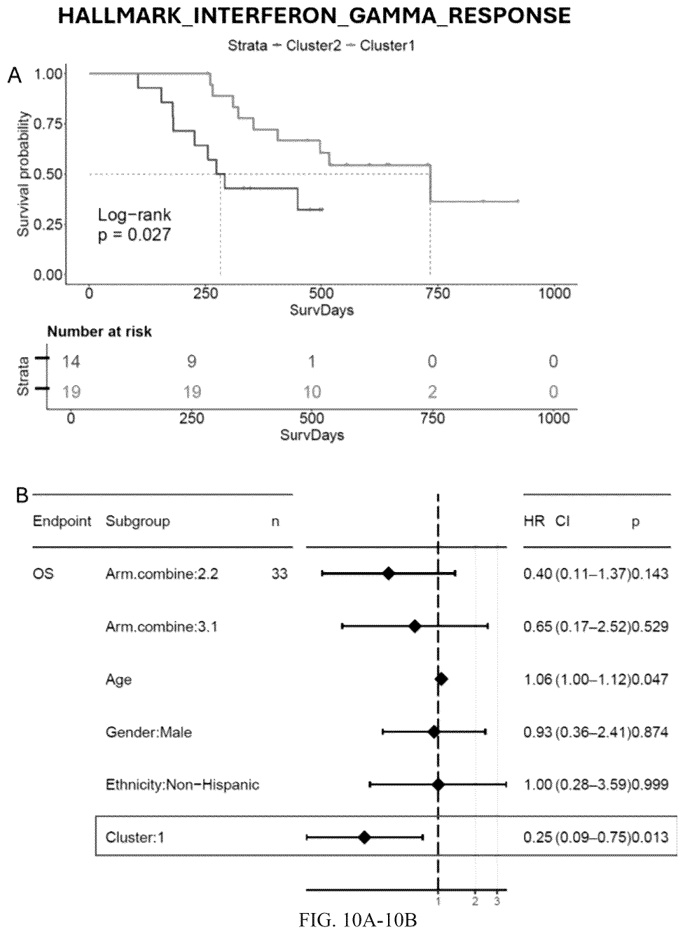

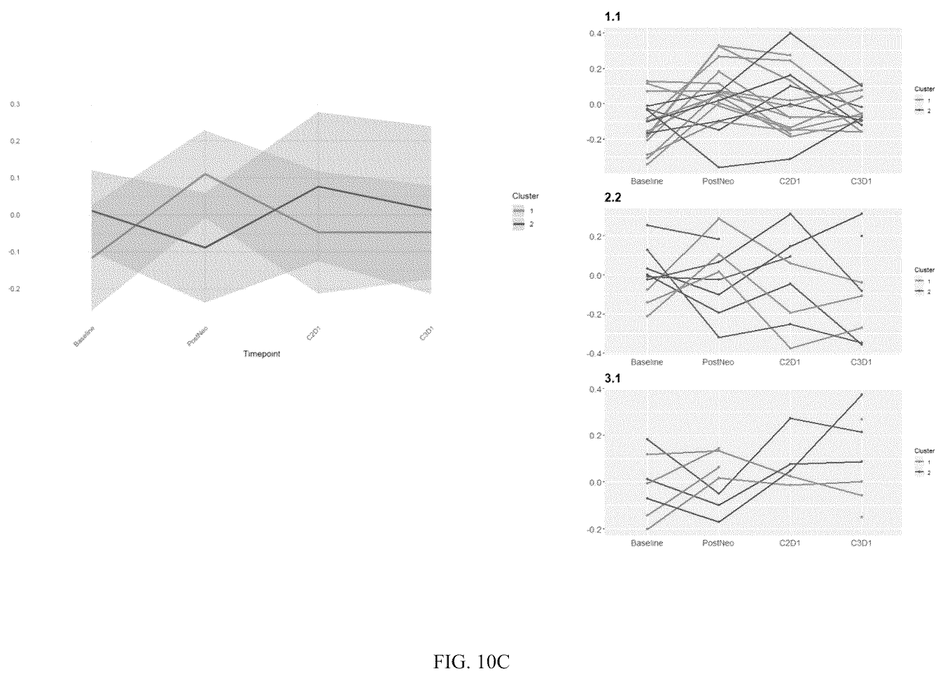

Finally, the inventors asked if the magnitude of interferon pathway induction by the adjuvant TLR agonist treatment directly correlated with OS or PFS. This could allow for the use of an interferon activity score as a biomarker for productive anti-tumor immune responses following ATL-DC immunotherapy. To this end, the inventors stratified the patients by the median GSVA score of the “HALLMARK INTERFERON GAMMA RESPONSE” gene set in their post-treatment PBMC samples. The inventors confirmed that patients whose post-treatment samples displayed higher interferon gene set scores (≥median) have longer OS and PFS than those with lower scores (FIG. 4A, FIG. 8A). Separate analyses on the grade IV (GBM) and grade III glioma patients showed a concordant trend but with a lower degree of statistical significance; this was likely caused by the small sample sizes. Notably, multivariate Cox PH analysis confirmed that the interferon gene set score is a significant predictor of tumor recurrence (FIG. 4B, C) and death (FIG. 8B). To ensure that the correlation is not specific to this single gene set, the inventors confirmed that the gene set scores of other interferon gene sets after ATL-DC treatment are also positively correlated with the patients OS and PFS (data not shown).

Taken together, these data suggest that the addition of TLR agonists to ATL-DC vaccination shifts towards an interferon-induced immune response in both lymphoid and myeloid cells. The magnitude of this induction may be correlated with the systemic immune response and clinical outcome.

### C. Discussion

The inventors report herein that ATL-DC vaccination with adjuvant poly-ICLC or resiquimod is overall safe and well-tolerated in patients with malignant glioma. To achieve the primary immunological endpoints of this study, the inventors utilized high-dimensional single-cell analysis to understand the systemic proteomic and transcriptomic changes induced by agonists of TLRs and other PRR in order to rationally determine the optimal therapeutic combination.

This study is the first high dimensional single-cell analysis done in a clinical trial for malignant glioma patients treated with dendritic cell vaccination and TLR agonists. Although this study was not designed to examine what happens in the tumor microenvironment, the results indicate that the inventors are able to sensitively detect systemic changes in the blood after intradermal autologous dendritic cell vaccination with and without TLR agonists. Adjuvant TLR agonist treatment promotes the expression of IFNα/β and IFNγ-induced genes on the peripheral lymphoid and myeloid cells, and GSEA further confirmed increased expression of the IFNa and IFNγ downstream genes, including IFNα/β-induced proteins ISG15 and STAT1. ISG15 stimulates IFNγ from lymphocytes (36) and negatively regulates IFNα/β signaling (37), and type I IFN maintenance of STAT1 expression induces IFNγ signaling (38). Other genes that were significantly upregulated by TLR agonist treatment include PARP9-DTX3L, and this heterodimer is also known to amplify interferon signalling. (39) The results support the conclusion that DC vaccination with poly-ICLC induces Type I and Type II IFN responses more effectively than with adjuvant resiquimod or a dendritic cell vaccine alone. Similar to the inventors' results, additional studies have identified poly-ICLC as the most effective TLR/PRR agonist when compared with others (40,41). The downstream effect of this signaling in the lymphoid compartment appears to be increased T-cell activity, as well as decreased T cell exhaustion phenotype. Together, these effects may enhance of the activity of tumor antigen specific T cells generated from an active vaccine.

It is also important to recognize that, in contrast to resiquimod and even plain poly-IC, poly-ICLC signals through various PRRs in addition to TLR3, consistent with its role as an authentic viral mimic. The poly-lysine stabilizer also functions as a transfection agent. Specifically, poly-lysine bursts the endosome through a proton sponge effect and releases the dsRNA into the cytoplasm, where it then preferentially activates MDA5, OAS, PKR and other cytoplasmic dsRNA dependent systems (21). Among the actions generated putatively through MDA5 are a further increase in Type 1 IFN, depression of MDSC, expansion of CD8 T cell populations through IL-15, CD8 targeting and infiltration of tumor through CXCL10, and a direct Type 1 IFN-dependent effect on tumor endothelium through VCAM-1 (42). These effects are best seen with systemic (intramuscular or intravenous) rather than local (subcutaneous) administration, as the inventors have done in the current study. Such adjuvant responses induced by poly ICLC may play a role in the longer-term maintenance of the immune responses generated by ATL-DC vaccination, but further studies are required to verify these findings.

While malignant gliomas are usually conceived of as a locoregional disease with essentially no capacity to spread outside the central nervous system, there has been a growing understanding of the role that systemic tissues play in priming, developing and/or suppressing an immune response in the brain. The catalog of known pervasive systemic immune deficits in glioblastoma patients is continually growing (43). The failure of immune checkpoint inhibitor therapy in malignant gliomas has led many to conclude that immune cells in the tumor microenvironment of cancers unresponsive to these checkpoint inhibitors may exist in an irreversible, terminally exhausted state (44,45). The generation of de novo tumor antigen-specific immune responses in the periphery that lead to new T-cell infiltration into the tumor microenvironment may be required to overcome this barrier (46). Dendritic cell vaccines are a robust example of an agent capable of mediating the initiation of such a T-cell response.

The inventors were able to detect an immunosuppressive phenotype in the myeloid cells of the placebo treatment cohort. Monocytes and DCs after ATL-DC/placebo vaccination showed upregulation of CLEC12A, which is a recently characterized inhibitory pattern recognition receptor expressed selectively on myeloid cells. This C-type lectin receptor is known to be downregulated following activation (47) and thought to control noninfectious inflammation (48). Gene expression of CLEC12A was absent in the ATL-DC/poly-ICLC group and only slightly increased in the ATL-DC/resiquimod group. The fraction of monocytes in the systemic circulation is known to be an important biomarker for the response to PD-1 checkpoint blockade immunotherapy (49). In conjunction with the inventors' other findings that the TLR agonists induced a higher fraction of CD14+classical monocytes in the blood, such data suggest that the combination of ATL-DC+TLR agonist with immune checkpoint blockade may be a rational choice. In fact, the inventors have now initiated a phase I trial combining ATL-DC+Poly-ICLC with pembrolizumab in recurrent glioblastoma patients (NCT04201873).

In conclusion, the inventors demonstrate that autologous dendritic cell vaccination plus TLR agonists in patients with malignant gliomas generates a systemic interferon activation signature in the peripheral blood that is correlated with overall survival. Although this was a randomized study, it was powered for immune biomarker analysis, not for survival. As such, the clinical efficacy outcomes should be interpreted with caution. Given the noted long-term survival with the adjuvant use of poly-ICLC with DC vaccination, particularly in the grade III cohort of patients, further clinical trials that incorporate these combinations of immunotherapeutic agents are warranted.

### D. Methods

### 1. Study Design.

This was a single-center, randomized, open-label multi-arm phase II clinical trial. The study protocols were approved by independent ethics committees and institution review boards as required. Patients were recruited and completed treatment between 2010 and 2014, with survival follow-up until the present date. All patients gave written informed consent before enrollment.

Twenty-three patients with high-grade WHO Grade III or IV gliomas were enrolled in this protocol. To be eligible for the primary cohort, patients had to be >18 years and have newly diagnosed or recurrent WHO Grade III or IV malignant glioma, as determined through central pathology review. For all patients, a Karnofsky Performance Score (KPS) of ≥60, adequate bone marrow, liver, and renal function, life expectancy of ≥8 weeks, no other prior malignancy within the last 5 years, no active viral infections, and sufficient resected tumor material to produce the autologous vaccine were required. All newly diagnosed patients underwent surgical resection followed by radiation and chemotherapy with temozolomide for 6 weeks, per standard of care. Patients in the recurrent setting proceeded to trial treatment, after recovery from surgery. All patients were scheduled to receive ATL-DC. Patients were then randomized to receive either placebo, resiquimod (topical 0.2%, 3M), or poly-ICLC (20 mcg/kg i.m., Oncovir) as an adjuvant to the DC vaccine. Patients underwent leukapheresis to obtain adequate numbers of PBMC for DC generation. For the study treatment, the inventors processed the resected tumor tissue into a lysate, then prepared and cryopreserved the autologous DCs as the inventors previously described (2,3). Patients were then treated with three intradermal injections of autologous tumor lysate-pulsed DC plus adjuvant TLRs/placebo on days 0, 14, and 28. Follow-up for patients was conducted at the study site for vital signs, KPS, hematology and serum chemistries, as well as neurological and physical examinations.

### 2. Clinical Assessments.

Safety was assessed on the basis of occurrence of adverse events, which were categorized according to the NCI Common Toxicity Criteria for Adverse Events v. 4.0. Safety assessments were performed on the day of vaccination and 1 week after each vaccination during the treatment phase, and every 2 months thereafter until tumor progression or death.

Anatomic MR images were acquired prior to DC+adjuvant treatment and at 2-month intervals for all patients using the standardized brain tumor imaging protocol (BTIP) (50), including three dimensional pre- and post-contrast T1-weighted images at 1-1.5 mm isotropic resolution, two-dimensional T2-weighted and T2-weighted fluid attenuated inversion recovery (FLAIR) images with 3-4 mm slice thickness and no interslice gap, and diffusion-weighted images with b=0, 500, and 1000 s/mm², 3-4 mm slice thickness and no interslice gap. Disease progression was determined using the modified RANO criteria (51). Additionally, post-hoc quantitative tumor volumetric analysis was performed using contrast-enhanced T1-weighted digital subtraction maps and segmentation techniques described previously (52-54). Briefly, linear registration was first performed between all images including contrast enhanced T1-weighted images and T2-weighted and/or FLAIR images to nonenhanced T1-weighted images using a 12-degree-of-freedom transformation and a correlation coefficient cost function. Next, intensity normalization and bias field correction was performed for both nonenhanced and contrast enhanced T1-weighted images, and voxel-by-voxel subtraction between normalized nonenhanced and contrast-enhanced T1-weighted images was performed. Image voxels with a positive (greater than zero) before-to-after change in normalized contrast enhancement signal intensity (i.e., voxels increasing in MR signal after contrast agent administration) within T2-weighted FLAIR hyperintense regions were isolated to create the final T1 subtraction maps. Estimates of tumor volume included areas of contrast enhancement on T1 subtraction maps including central necrosis (defined as being enclosed by contiguous, positive enhancing disease).

### 3. Patient Samples.

Heparinized peripheral blood was collected at the baseline visit and at each treatment visit for immune monitoring. Peripheral blood mononuclear cells were collected in CPT tubes (BD Biosciences, cat: 362753), isolated according to the manufacturer's protocol, placed in freezing media made of 90% human AB serum (Fisher Scientific, cat. MT35060CI) and 10% dimethyl sulfoxide (Sigma, cat. C6295-50ML) and stored in liquid nitrogen until the time of analysis. On the day of data acquisition, samples were thawed in a 37° C. water bath and washed in RPMI-1640 media (Genesse Scientific, cat: 25-506) supplemented with FBS and penicillin and streptomycin. Patient tumor samples were attained immediately following surgery.

### 4. Generation of Autologous Dendritic Cell Vaccines.

Monocyte-derived DCs were established from adherent peripheral blood mononuclear cells (PBMC) obtained via leukapheresis performed at the UCLA Hemapheresis Unit, as the inventors have published on previously (3,6,55). All ex vivo DC preparations were performed in the UCLA-Jonsson Cancer Center GMP facility under sterile and monitored conditions. In brief, dendritic cells were prepared by culturing adherent cells from peripheral blood in RPMI-1640 (Gibco) and supplemented with 10% autologous serum, 500 U/mL GM-CSF (Leukine®, Amgen, Thousand Oaks, CA) and 500 U/mL of IL-4 (CellGenix), using techniques described previously (56). Following culture, DCs were collected by vigorous rinsing and washed with sterile 0.9% NaCl solution. The purity and phenotype of each DC lot was also determined by flow cytometry (FACScan flow cytometer; BD Biosciences, San Jose, CA). Cells were stained with FITC-conjugated CD83, PE-conjugated CD86 and PerCP-conjugated HLA-DR mAb's (BD Biosciences). Release criteria were >70% viable by trypan blue exclusion, and >30% of the large cell gate being CD86+ and HLA-DR+. One day before each vaccination, DC were pulsed (co-cultured) with tumor lysate overnight, washed, and the final product was tested for sterility by Gram stain, Mycoplasma and endotoxin testing prior to injection.

### 5. Molecular and Immune Analyses

CyTOF. Cells for mass cytometry analysis were prepared according to the Maxpar cell surface staining protocol. Briefly, 0.5 to 3×10⁶ cells were washed with PBS and treated with 0.1 mg/mL of DNAse I Solution (StemCell Technologies, cat: 07900) for 15 minutes at room temperature. Cells were then resuspended in 5 μM Cell-ID cisplatin (Fluidigm, cat: 201064) as a live/dead marker for 5 minutes at room temperature. After quenching with the Maxpar cell staining buffer (Fluidigm, cat: 201068), the cells were incubated with a 23-marker panel for 30 minutes at room temperature. After washing, cells were incubated overnight in 125 nM iridium intercalation solution (1000× dilution of 125 μM Cell-ID Intercalator-Ir; Fluidigm, cat: 201192A) in Maxpar Fix and Perm Buffer (Fluidigm, cat: 201067) to label intracellular DNA. The next morning, cells were washed with cell staining buffer and distilled water. The samples were processed on a Helios mass cytometer (Fluidigm) in the University of California, Los Angeles Jonsson Comprehensive Cancer Center Flow Cytometry core.

The CyTOF data was normalized utilizing EQ four element calibration beads (Fluidigm, cat: 201078) with the R package premessa (version 0.2.4, Parker Institute for Cancer Immunotherapy) following removal of dead cells. A total of 5,000 cells were subsampled from each sample (except for sample S16-07-2-Day 1 where the inventors only had 4,861 cells). Subsequently, bead normalized data from 45 samples were integrated as described previously (57). Briefly, flow cytometry standard (FCS) files were loaded into R with the flowCore package (version 2.8.0). Raw marker intensities were transformed utilizing hyperbolic inverse sine (arc sin h) with cofactor of 5. Cell population identification was carried out using unsupervised clustering using FlowSOM package (version 2.4.0) and subsequent metaclustering using ConsensusClusterPlus package (version 1.60.0). The metaclusters were manually curated to identify canonical populations in FIG. 2B (including one unknown cluster with little/no marker expression). The high dimensional data was visualized with the Uniform Manifold Approximation and Projection (UMAP). Differential marker analysis across treatment groups were first performed using the linear mixed model analysis pipeline as described (57). Markers with nominally significant p-values in one or more cell populations (P≤0.05; e.g CD39, CD38, Ki-67, PD-1) were visualized in boxplots; statistical significance computed using the linear mixed model were further confirmed using non-parametric Wilcoxon rank sum test.

Bulk RNAseq. Total RNA was isolated from frozen PBMC of the patients isolated at baseline and after three biweekly vaccines with ATL-DC plus adjuvant (placebo: 5 pairs, resiquimod: 8 pairs and poly-ICLC: 8 pairs; see Supplementary Table Clinical) using the ZYMO quick RNA extraction kit. The inventors utilized the TruSeq RNA exome kit to construct the RNA sequencing libraries. Paired-end, 2×100 base pair (bp) transcriptome reads were mapped to the Genome Reference Consortium Human Build 38 (GRCh38) reference genome using HISAT2 (version 2.0.6) (58). The gene level counts were generated by the HTSeq-count program (version 0.5.4p5) (59). The inventors utilized the DESeq2 R package's counts function (version 1.24.0) (60) to compute the normalized gene expression values from the raw gene expression counts. DESeq2 normalized gene expression was log 2 transformed after adding a pseudo count of 1. For subsequent differentially expressed genes (DEGs) and gene set enrichment analyses, the inventors only included the known genes (i.e genes with RefSeq transcripts ID starting with “NM_”, that satisfy: 1) normalized expression IQR≥1; and 2) normalized log 2 expression ≥1 in at least one of the samples.

Based on the filtered gene list, the inventors first obtained the patient-specific, log 2 fold change of each gene before and after the ATL-DC vaccine treatment. Next, the mean of the log 2 fold changes in the poly-ICLC or resiquimod group is compared to those in the placebo group. Genes showing at least 2-fold upregulation in any of the TLR agonist treated group (resiquimod or poly-ICLC, nominal t-test p-value ≤0.05) with respect to the placebo were tested for significant overlap with gene ontology and known gene sets using the web-based tools, ENRICHR (61).

To calculate single sample gene set enrichment of the interferon related genes, the inventors used the Gene Set Variation Analysis (GSVA) package (version 1.32.0) (62). To compute the GSVA scores, the filtered, log 2 normalized gene expression were supplied to the GSVA program using the ‘kcdf=Gaussian’ mode. The inventors manually selected gene sets that are related to interferon pathway activation from the c2.cgp, c6, c7, hallmark geneset collections of the Broad Institute's Molecular Signatures Database (version 7.0) (63).

Single-cell RNA-seq sample processing and data analysis. The cells for scRNAseq analysis were resuspended in PBS at a concentration of 1,000 cells/μl. The inventors only selected representative patients from each treatment group whose PBMC quality were sufficient for single cell RNAseq processing. Cell preparation, library preparation, and sequencing were carried out according to Chromium product-based manufacturer protocols (10× Genomics), targeting for a total of 10,000 cells sequenced. Single cell RNA sequencing was carried out on a Novaseq 6000 S2 2×50 bp flow cell (Illumina) utilizing the Chromium single cell 3′ gene expression library preparation (10× Genomics).

The data was aligned with Cell Ranger (version 3.1.0) and aligned to the Genome Reference Consortium Human Build 38 (GRCh38). Data was imported into R (version 4.2.1) and analyzed with the Seurat package (version 4.2.0) (64). For quality assurance, cells with greater than 20% mitochondrial features were excluded from further analysis. The inventors analyzed a total of 99,590 cells after the QC step. The Seurat data object from each sample were then integrated and scaled, regressing out the percent mitochondrial features and cell cycle score difference, as described (see, for example: satijalab.org/seurat/index.html). The inventors manually identified each cluster using the genes that were differentially expressed as determined by FindAllMarker function; they are visualized using R's ggplot2 and pheatmap packages. Differentially expressed genes (DEGs) corresponding to each treatment group (Placebo vs. Poly-ICLC vs. resiquimod) were computed by first computing cluster-specific DEGs between each group against the pre-treatment (Day 0) samples. The union of cluster-specific DEGs that were seen in at least 20% of all comparisons (the total number of comparisons is the number of treatment groups (3 groups) times number of lymphoid or myeloid clusters) were selected as recurrent DEGs shown in the heatmaps of FIGS. 2F and 2G.

### 6. Statistical Analysis.

For the percentage comparisons in the CyTOF analysis, the inventors used the Wilcoxon rank sum test for non-parametric data for 2 independent samples and compared baseline (Day 0) to Day 1 or Day 29. The inventors performed Fisher's exact test for testing the null of independence of the phenotypic and genotypic characteristics and treatments using the stats package in R. Differences in transcript expression log 2 fold changes FC and GSVA scores in the bulk RNA-seq data were calculated with unpaired T test with non-equal variances (two-sided Welch t test). The differences in overall survival or time to progression following treatment (either combination of ATL-DC and placebo, ATL-DC and adjuvant poly-ICLC or ATL-DC and adjuvant resiquimod treatment) were assessed using log-rank test (visualized using R's survminer package). The inventors further performed multivariable cox proportional hazard (cox PH) regression analysis with HRs (95% CIs) to determine if any of the treatment regimen were significantly predictive of overall survival or time to progression after adjusting for clinical covariates, such as WHO grade, number of recurrences, and MGMT status. The association between interferon pathway score and overall survival or time to progression was analyzed similarly using log-rank (univariate) and Cox PH (multivariate) analyses.

### E. Tables

TABLE 1
Baseline patient characteristics.

| | DC vaccine + | | DC vaccine + | | DC vaccine + | | | |
|---|---|---|---|---|---|---|---|---|
| | placebo | | poly-ICLC | | resiquimod | | Total | |
| Variable | (n = 5) | | (n = 9) | | (n = 9) | | (n = 23) | |
| Age (year) | | | | | | | | |
| Mean (SD) | 56.50 | (13.75) | 44.09 | (12.04) | 43.73 | (8.80) | 46.65 | (11.98) |
| Median (IQR) | 48.06 | | 40.15 | | 43.46 | | 45.33 | |
| Sex | | | | | | | | |
| Female, n (%) | 2 | (40%) | 5 | (56%) | 3 | (33%) | 10 | (43%) |
| Male, n (%) | 3 | (60%) | 4 | (44%) | 6 | (67%) | 13 | (57%) |
| OS (months) | | | | | | | | |
| Mean (SD) | 19.51 | (23.49) | 54.06 | (34.63) | 28.05 | (23.82) | 36-39 | (30-93) |
| Median (iQr) | 7.70 | | 52.50 | | 16.73 | | 24.47 | |
| TTP (months) | | | | | | | | |
| Mean (SD) | 5.06 | (1.10) | 44.64 | (36.26) | 10.96 | (8.64) | 22.86 | (28.80) |
| Median (IQR) | 5.13 | | 31.43 | | 8.10 | | 8.10 | |
| WHO grade, n (%) | | | | | | | | |
| III | 1 | (20%) | 4 | (44%) | 3 | (33%) | 7 | (30%) |
| IV | 4 | (80%) | 5 | (56%) | 7 | (78%) | 16 | (70%) |
| Recurrence, n (%) | | | | | | | | |
| None | 1 | (20%) | 5 | (56%) | 3 | (33%) | 9 | (39%) |
| Recurrence | 4 | (80%) | 4 | (44%) | 6 | (67%) | 14 | (61%) |
| MGMT status, n (%) | | | | | | | | |
| Methylated | 1 | (20%) | 4 | (44%) | 3 | (33%) | 8 | (35%) |
| Unmethylated | 4 | (80%) | 5 | (56%) | 6 | (67%) | 15 | (65%) |
| EGFR | | | | | | | | |
| classification, n (%) | | | | | | | | |
| Amplified | 3 | (60%) | 2 | (22%) | 5 | (56%) | 10 | (44%) |
| Not amplified | 1 | (20%) | 5 | (56%) | 3 | (33%) | 9 | (39%) |
| Unknown | 1 | (20%) | 2 | (22%) | 1 | (11%) | 4 | (17%) |
| IDH status, n (%) | | | | | | | | |
| Mutant | 1 | (20%) | 4 | (44%) | 3 | (33%) | 8 | (35%) |
| Wild-type | 4 | (80%) | 5 | (56%) | 6 | (67%) | 15 | (65%) |
| Avastin treatment, n (%) | | | | | | | | |
| Treated | 4 | (80%) | 5 | (56%) | 6 | (67%) | 15 | (65%) |
| Not treated | 1 | (20%) | 4 | (44%) | 3 | (33%) | 8 | (35%) |

TABLE 2
Adverse events across treatment cohorts.

| | DC | DC | DC | |
|---|---|---|---|---|
| | vaccine + | vaccine + | vaccine + | |
| | placebo | poly-ICLC | resiquimod | Total |
| Variable | (n = 5) | (n = 9) | (n = 9) | (n = 23) |
| Any | 1 (20%) | 9 (100%) | 8 (89%) | 18 (78%) |
| Rash | 0 | 1 (11%) | 8 (89%) | 9 (39%) |
| Fever | 0 | 5 (56%) | 3 (33%) | 8 (35%) |
| Fatigue | 1 (20%) | 2 (22%) | 3 (33%) | 6 (26%) |
| Flu-like | 0 | 2 (22%) | 0 | 2 (9%) |
| symptoms | | | | |
| Nasal | 0 | 1 (11%) | 0 | 1 (4%) |
| congestion | | | | |
| Nervous system | 0 | 4 (44%) | 2 (22%) | 4 (17%) |
| Headache | 0 | 3 (33%) | 1 (11%) | 4 (17%) |
| Seizure | 0 | 0 | 1 (11%) | 1 (4%) |
| Sensory | 0 | 1 (11%) | 0 | 1 (4%) |
| paresthesias | | | | |
| Cognitive | 0 | 1 (11%) | 0 | 1 (4%) |
| disturbances | | | | |
| Ear pain | 0 | 1 (11%) | 0 | 1 (4%) |
| Musculoskeletal | 0 | 3 (33%) | 2 (22%) | 5 (22%) |
| Neck pain | 0 | 0 | 1 (11%) | 1 (4%) |
| Body aches | 0 | 1 (11%) | 0 | 1 (4%) |
| Myalgia | 0 | 2 (22%) | 1 (11%) | 3 (13%) |
| Gastrointestinal | 0 | 1 (11%) | 1 (11%) | 2 (9%) |
| Nausea | 0 | 1 (11%) | 1 (11%) | 2 (9%) |
| Vomiting | 0 | 0 | 1 (11%) | 1 (4%) |
| Cardiovascular/ | 0 | 0 | 2 (22%) | 2 (9%) |
| blood | | | | |
| Presyncope | 0 | 0 | 1 (11%) | 1 (4%) |
| Neutropenia | 0 | 0 | 1 (11%) | 1 (4%) |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the references cited throughout the specification, to the extent that they provide exemplary procedural or other details supplementary to those set forth herein, are specifically incorporated herein by reference.
1. Roger Stupp M. E. H., Mason Warren P, van den Bent Martin J, Taphoorn Martin J B, Janzer Robert C, Ludwin Samuel K, Allgeier Anouk, Fisher Barbara, Belanger Karl, Hau Peter, Brandes Alba A, Gijtenbeck Johanna, Marosi Christine, Vecht Charles J, Mokhtari Karima, Wesseling Pieter, Villa Salvador, Eisenhauer Elizabeth, Gorlia Thierry, Weller Michael, Lacombe Denis, Gregory Cairncross J, Mirimanoff Rene-Olivier. Effects of radiotherapy with concomitant and adjuvant temozolomide versus radiotherapy alone on survival in glioblastoma in a randomised phase III study: 5-year analysis of the EORTC-NCIC trial. Lancet Oncol 10, 459-466 (2009).
2. Linda M. Liau R. M. P., Kiertscher Sylvia M., Odesa Sylvia K, Kremen Thomas J, Giovannone Adrian J., Lin Jia-Wei, Chute Dennis J., Mischel Paul S., Cloughesy Timothy F., and Roth Michael D. Dendritic Cell Vaccination in Glioblastoma Patients Induces Systemic and Intracranial T-cell Responses Modulated by the Local Central Nervous System Tumor Microenvironment. Clin Cancer Res 11, 5515-5525 (2005).
3. Prins R. M., et al. Gene expression profile correlates with T-cell infiltration and relative survival in glioblastoma patients vaccinated with dendritic cell immunotherapy. Clin Cancer Res 17, 1603-1615 (2011).
4. Okada H., et al. Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol 29, 330-336 (2011).
5. Liau Linda M., et al. First results on survival from a large Phase 3 clinical trial of an autologous dendritic cell vaccine in newly diagnosed glioblastoma. J Transl Med 16, 1-9 (2018).
6. Liau L. M., et al. Association of Autologous Tumor Lysate-Loaded Dendritic Cell Vaccination With Extension of Survival Among Patients With Newly Diagnosed and Recurrent Glioblastoma: A Phase 3 Prospective Externally Controlled Cohort Trial. JAMA Oncol 9, 112-121 (2023). [PMC free article] [PubMed] [Google Scholar]
7. Yu M. & Levine S. J. Toll-like receptor, RIG-I-like receptors and the NLRP3 inflammasome: key modulators of innate immune responses to double-stranded RNA viruses. Cytokine Growth Factor Rev 22, 63-72 (2011).
8. Medzhitov R. Recognition of microorganisms and activation of the immune response. Nature 449, 819-826 (2007).
9. Huang B., Zhao J., Unkeless J. C., Feng Z. H. & Xiong H. TLR signaling by tumor and immune cells: a double-edged sword. Oncogene 27, 218-224 (2008).
10. Wang R. F., Miyahara Y. & Wang H. Y. Toll-like receptors and immune regulation: implications for cancer therapy. Oncogene 27, 181-189 (2008).
11. Kawasaki T. & Kawai T. Toll-like receptor signaling pathways. Front Immunol 5, 461 (2014).
12. Matsumoto M., et al. Subcellular localization of Toll-like receptor 3 in human dendritic cells. J Immunol 171, 3154-3162 (2003).
13. Seya T., Funami K., Taniguchi M. & Matsumoto M. Antibodies against human Toll-like receptors (TLRs): TLR distribution and localization in human dendritic cells. J Endotoxin Res 11, 369-374 (2005).
14. McKimmie C. S. & Fazakerley J. K. In response to pathogens, glial cells dynamically and differentially regulate Toll-like receptor gene expression. J Neuroimmunol 169, 116-125 (2005).
15. Olson J. K. & Miller S. D. Microglia initiate central nervous system innate and adaptive immune responses through multiple TLRs. J Immunol 173, 3916-3924 (2004).
16. Carpentier P. A., et al. Differential activation of astrocytes by innate and adaptive immune stimuli. Glia 49, 360-374 (2005).
17. Farina C., et al. Preferential expression and function of Toll-like receptor 3 in human astrocytes. J Neuroimmunol 159, 12-19 (2005).
18. Bsibsi M., et al. Toll-like receptor 3 on adult human astrocytes triggers production of neuroprotective mediators. Glia 53, 688-695 (2006).
19. Zhu X., et al. Toll like receptor-3 ligand poly-ICLC promotes the efficacy of peripheral vaccinations with tumor antigen-derived peptide epitopes in murine CNS tumor models. J Transl Med 5, 10 (2007).
20. Guarda G., et al. Type I interferon inhibits interleukin-1 production and inflammasome activation. Immunity 34, 213-223 (2011).
21. Sultan H., Wu J., Kumai T., Salazar A. M. & Celis E. Role of MDA5 and interferon-I in dendritic cells for T cell expansion by anti-tumor peptide vaccines in mice. Cancer Immunol Immunother 67, 1091-1103 (2018).
22. Zhu X., et al. Poly-ICLC promotes the infiltration of effector T cells into intracranial gliomas via induction of CXCL10 in IFN-alpha and IFN-gamma dependent manners. Cancer Immunol Immunother 59, 1401-1409 (2010).
23. Kyi C., et al. Therapeutic Immune Modulation against Solid Cancers with Intratumoral Poly-ICLC: A Pilot Trial. Clin Cancer Res 24, 4937-4948 (2018).
24. Giantonio B. J., et al. Toxicity and response evaluation of the interferon inducer poly ICLC administered at low dose in advanced renal carcinoma and relapsed or refractory lymphoma: a report of two clinical trials of the Eastern Cooperative Oncology Group. Invest New Drugs 19, 89-92 (2001).
25. Salazar A. M., et al. Long-term treatment of malignant gliomas with intramuscularly administered polyinosinic-polycytidylic acid stabilized with polylysine and carboxymethylcellulose: an open pilot study. Neurosurgery 38, 1096-1103; discussion 1103-1094 (1996).
26. Butowski N., et al. A phase II clinical trial of poly-ICLC with radiation for adult patients with newly diagnosed supratentorial glioblastoma: a North American Brain Tumor Consortium (NABTC01-05). J Neurooncol 91, 175-182 (2009).
27. Smits E. L., Ponsaerts P., Berneman Z. N. & Van Tendeloo V. F. The use of TLR7 and TLR8 ligands for the enhancement of cancer immunotherapy. Oncologist 13, 859-875 (2008).
28. Prins R. M., et al. The TLR-7 agonist, imiquimod, enhances dendritic cell survival and promotes tumor antigen-specific T cell priming: relation to central nervous system antitumor immunity. J Immunol 176, 157-164 (2006).
29. Chang B. A., Cross J. L., Najar H. M. & Dutz J. P. Topical resiquimod promotes priming of CTL to parenteral antigens. Vaccine 27, 5791-5799 (2009).
30. Du J., et al. TLR8 agonists stimulate newly recruited monocyte-derived cells into potent APCs that enhance HBsAg immunogenicity. Vaccine 28, 6273-6281 (2010).
31. Rajagopal D., et al. Plasmacytoid dendritic cell-derived type I interferon is crucial for the adjuvant activity of Toll-like receptor 7 agonists. Blood 115, 1949-1957 (2010).
32. Nair S., et al. Injection of immature dendritic cells into adjuvant-treated skin obviates the need for ex vivo maturation. J Immunol 171, 6275-6282 (2003).
33. Canale F. P., et al. CD39 Expression Defines Cell Exhaustion in Tumor-Infiltrating CD8+ T Cells. Cancer Research 78, 115-128 (2018).
34. Philip M., et al. Chromatin states define tumour-specific T cell dysfunction and reprogramming. Nature 545, 452-456 (2017).
35. Vignali P. D. A., et al. Hypoxia drives CD39-dependent suppressor function in exhausted T cells to limit antitumor immunity. Nature Immunology 24, 267-279 (2023).
36. Swaim C. D., Scott A. F., Canadeo L. A. & Huibregtse J. M. Extracellular ISG15 Signals Cytokine Secretion through the LFA-1 Integrin Receptor. Mol Cell 68, 581-590 e585 (2017).
37. Zhang X., et al. Human intracellular ISG15 prevents interferon-alpha/beta over-amplification and auto-inflammation. Nature 517, 89-93 (2015).
38. Gough D. J., et al. Functional crosstalk between type I and II interferon through the regulated expression of STAT1. PLOS Biol 8, e1000361 (2010).
39. Zhang Y., et al. PARP9-DTX3L ubiquitin ligase targets host histone H2BJ and viral 3C protease to enhance interferon signaling and control viral infection. Nat Immunol 16, 1215-1227 (2015).
40. Park H., et al. Polyinosinic-polycytidylic acid is the most effective TLR adjuvant for SIV Gag protein-induced T cell responses in nonhuman primates. J Immunol 190, 4103-4115 (2013).
41. Saxena M., et al. Poly-ICLC, a TLR3 Agonist, Induces Transient Innate Immune Responses in Patients With Treated HIV-Infection: A Randomized Double-Blinded Placebo Controlled Trial. Front Immunol 10, 725 (2019).
42. Sultan H., et al. Poly-IC enhances the effectiveness of cancer immunotherapy by promoting T cell tumor infiltration. J Immunother Cancer 8 (2020).
43. Ott M., Prins R. M. & Heimberger A. B. The immune landscape of common CNS malignancies: implications for immunotherapy. Nat Rev Clin Oncol 18, 729-744 (2021).
44. Jiang W., et al. Exhausted CD8+T Cells in the Tumor Immune Microenvironment: New Pathways to Therapy. Front Immunol 11, 622509 (2020).
45. Reardon D. A., et al. Effect of Nivolumab vs Bevacizumab in Patients With Recurrent Glioblastoma: The CheckMate 143 Phase 3 Randomized Clinical Trial. JAMA Oncol 6, 1003-1010 (2020).
46. Hiam-Galvez K. J., Allen B. M. & Spitzer M. H. Systemic immunity in cancer. Nat Rev Cancer 21, 345-359 (2021).
47. Marshall A. S. J., et al. Human MICL (CLEC12A) is differentially glycosylated and is downregulated following cellular activation. European Journal of Immunology 36, 2159-2169 (2006).
48. Neumann K., et al. Clec 12a is an inhibitory receptor for uric acid crystals that regulates inflammation in response to cell death. Immunity 40, 389-399 (2014). [
49. Krieg C., et al. High-dimensional single-cell analysis predicts response to anti-PD-1 immunotherapy. Nature Medicine 24, 144-153 (2018).
50. Ellingson B. M., et al. Consensus recommendations for a standardized Brain Tumor Imaging Protocol in clinical trials. Neuro Oncol 17, 1188-1198 (2015).
51. Wen P. Y., et al. Updated response assessment criteria for high-grade gliomas: response assessment in neuro-oncology working group. J Clin Oncol 28, 1963-1972 (2010).
52. Ellingson B. M., et al. Volumetric response quantified using T1 subtraction predicts long-term survival benefit from cabozantinib monotherapy in recurrent glioblastoma. Neuro Oncol 20, 1411-1418 (2018).
53. Ellingson B. M., et al. Recurrent glioblastoma treated with bevacizumab: contrast-enhanced T1-weighted subtraction maps improve tumor delineation and aid prediction of survival in a multicenter clinical trial. Radiology 271, 200-210 (2014).
54. Ellingson B. M., et al. Contrast-enhancing tumor growth dynamics of preoperative, treatment-naive human glioblastoma. Cancer 122, 1718-1727 (2016).
55. Prins R. M., Cloughesy T. F. & Liau L. M. Cytomegalovirus Immunity after Vaccination with Autologous Glioblastoma Lysate. New England Journal of Medicine 359, 539-541 (2008).
56. Liau L. M., et al. Dendritic cell vaccination in glioblastoma patients induces systemic and intracranial T-cell responses modulated by the local central nervous system tumor microenvironment. Clin Cancer Res 11, 5515-5525 (2005).
57. Nowicka M., et al. CyTOF workflow: differential discovery in high-throughput high-dimensional cytometry datasets. F1000Res 6, 748 (2017).
58. Kim D., Langmead B. & Salzberg S. L. HISAT: a fast spliced aligner with low memory requirements. Nat Methods 12, 357-360 (2015).
59. Anders S., Pyl P. T. & Huber W. HTSeq—a Python framework to work with high-throughput sequencing data. Bioinformatics 31, 166-169 (2015).
60. Love M. I., Huber W. & Anders S. Moderated estimation of fold change and dispersion for RNA-seq data with DESeq2. Genome Biol 15, 550 (2014).
61. Kuleshov M. V., et al. Enrichr: a comprehensive gene set enrichment analysis web server 2016 update. Nucleic Acids Res 44, W90-97 (2016).
62. Hanzelmann S., Castelo R. & Guinney J. GSVA: gene set variation analysis for microarray and RNA-seq data. BMC Bioinformatics 14, 7 (2013
63. Subramanian A., et al. Gene set enrichment analysis: a knowledge-based approach for interpreting genome-wide expression profiles. Proc Natl Acad Sci USA 102, 15545-15550 (2005).
64. Butler A., Hoffman P., Smibert P., Papalexi E. & Satija R. Integrating single-cell transcriptomic data across different conditions, technologies, and species. Nat Biotechnol 36, 411-420 (2018).

1. A method for treating a subject for brain cancer, the method comprising administering to the subject an immunotherapy, wherein the subject has been evaluated for an interferon activation response in a biological sample from the subject.

2. The method of claim 1, wherein the brain cancer comprises glioma.

3-8. (canceled)

9. The method of claim 1, wherein the subject has previously been treated with an anti-cancer therapy and wherein the anti-cancer therapy comprises a TLR agonist, neoadjuvant therapy, and/or an immunotherapy.

10. (canceled)

11. The method of claim 1, wherein the subject has been treated, will be treated, or is being treated with one or both of a TLR agonist and an immunotherapy and wherein the immunotherapy comprises a cancer vaccine.

12. (canceled)

13. The method of claim 11, wherein the cancer vaccine comprises a dendritic cell (DC) vaccine or neoantigen therapy.

14. The method of claim 13, wherein the dendritic cell vaccine comprises an autologous tumor lysate (ATL) DC vaccine.

15-16. (canceled)

17. The method of claim 1, wherein the immunotherapy comprises immune checkpoint inhibitor (ICI) therapy and wherein the ICI therapy comprises an inhibitor of PD-1, PDL1, PDL2, and/or CTLA-4.

18-21. (canceled)

22. The method of claim 17, wherein the ICI therapy comprises ipilimumab and nivolumab.

23. The method of claim 11, wherein the immunotherapy is an adjuvant therapy.

24. The method of claim 9, wherein the TLR agonist comprises a TLR3, TLR7, TLR8, and/or TLR7/8 TLR agonist.

25. The method of claim 24, wherein the TLR agonist comprises a TLR3 agonist and wherein the TLR3 agonist comprises polyinosinic acid polycytidylic acid stabilized with polylysine (poly-ICLC).

26. (canceled)

27. The method of claim 24, wherein the TLR agonist comprises a TLR7 or TLR7/8 agonist.

28. The method of claim 27, wherein the TLR agonist comprises Imiquimod or Resiquimod.

29. The method of claim 1, wherein the biological sample comprises peripheral blood mononuclear cells (PBMCs).

30. The method of claim 1, wherein the interferon activation response comprises a GSVA score of greater than 0 for a IFNg gene set or IFNa gene set.

31. (canceled)

32. The method of claim 1, wherein the biological sample is from a subject that received a cancer vaccine at a time point of 0-24 hours prior to the collection of the biological sample.

33. The method of claim 30, wherein the GSVA score is a score based on the interferon gene set of Table 3, 4, or 5, or any combinations of genes therein.

34. A method for: (i) predicting subject outcomes and/or for predicting the effectiveness of an immunotherapy for treating brain cancer in a subject in need thereof; (ii) monitoring a subject having brain cancer and being treated with an immunotherapy; or (iii) evaluating a subject having brain cancer; the method comprising determining an interferon activation response from a biological sample from the subject.

35-97. (canceled)

98. A method for treating a subject having brain cancer and that has been previously treated with a neoadjuvant cancer vaccine, the method comprising administering an adjuvant immunotherapy to the subject; wherein the subject has been determined to have a positive interferon activation response in a biological sample from the subject; wherein the immunotherapy comprises immune checkpoint inhibitor (ICI) therapy; and wherein the cancer vaccine comprises ATL-DC vaccine or neoantigen therapy.

99. The method of claim 1, wherein the subject is a human subject.
